(19) United States
(12) Patent Application Publication
(10) Pub. No.: US 2010/0197657 A1
(43) Pub. Date: Aug. 5, 2010

(54) 2-ARYL OR HETEROARYL INDOLE DERIVATIVES

(76) Inventors: Ronald K. Chang, Ambler, PA (US); Christina Ng Di Marco, Conshohocken, PA (US); Scott D. Kuduk, Harleysville, PA (US)

Correspondence Address: MERCK, P O BOX 2000, RAHWAY, NJ, 07065-0907, US

(21) Appl. No.: 12/677,242

(22) PCT Filed: Sep. 22, 2008
(86) PCT No.: PCT/US08/10970

§ 371 Date: Mar. 9, 2010

Related U.S. Application Data
(60) Provisional application No. 60/995,149, filed on Sep. 25, 2007.

(51) Int. Cl. A61K 31/553; C07D 209/14; A61K 31/404; C07D 401/04; A61K 31/4439; A61K 31/4523; C07D 413/12; A61K 31/5377; C07D 267/14; A61P 25/02; A61P 35/00; A61P 25/22; A61P 3/04; A61P 1/00; A61P 3/14; A61P 9/00

(52) U.S. Cl. 514/211.09; 548/503; 514/415; 546/277.4; 514/339; 514/323; 546/201; 544/143; 514/235.2; 540/552

(57) ABSTRACT

The present invention provides 2-aryl or heteroaryl indole derivatives which are ASIC channel modulators, maceutical compositions containing such compounds, and methods of using them as therapeutic agents.

### CROSS REFERENCE TO RELATED APPLICATIONS

This application claims the benefit of U.S. Provisional Application No. 60/995,149, filed Sep. 25, 2007, the contents of which are incorporated herein by reference in their entirety.

### BACKGROUND OF THE INVENTION

Under pathological conditions the release of protons can exceed the organism's ability to buffer extracellular pH and tissue acidification ensues. In mammals, this decrease in extracellular pH can trigger neuronal activity through the binding of protons to receptors on the surface of nearby neurons. Acid-sensing ion channels, or ASICs, represent a major known detector for protons in both central and peripheral neurons. ASICs belong to a family of ion channels related to the degenerins of nematodes and mammalian epithelial Na+ channels (ENaC)(Physiol Rev (2002) 82, 735-767). The tertiary structure of ASIC's pore-forming subunits is predicted to span the plasma membrane twice with the majority of amino acids in the extracellular domain. Activation of ASICs by protons leads to influx of Na+, depolarization, and neuronal excitation. ASICs are principally found in neurons, although some somatic expression has been demonstrated.

The pharmacology of ASIC channels is limited. Amiloride and its analog, benzamil, are non-selective inhibitors of most members of the DEG\ENaC family (Physiol Rev (2002) 82, 735-767). Although amiloride and its analogs are not potent ASIC channel blockers, they show efficacy in vivo. Two peptide toxins, Psalmotoxin-1 and APETx2, were isolated from animal venoms and are selective inhibitors of ASIC1 and ASIC3, respectively. These toxins inhibit ASIC current at nM concentrations and have demonstrated utility in understanding ASIC function in animals (Toxicon (2007) 49, 271-284). A nonselective, small-molecule ASIC inhibitor (A-317567) has been recently developed with a >10-fold increase in potency vs. amiloride in in vivo and in vitro assays (Pain (2005) 117, 88-96). The invertebrate neuropeptide FMRFamide, increases activity of some members of the DEG/ENaC family including ASIC (Peptides (2006) 27, 1138-1152). ASIC3 current is transient in the absence of FMRFamide due to acute desensitization. In the presence of FMRFamide, desensitization is profoundly slowed, resulting in a large potentiation of the proton-evoked current and a dramatic increase in steady-state current. Other structurally related peptides show similar activities. Little effect of FMRFamide related peptides is observed in ASIC channels without ASIC3 subunits.

Invertebrate members of the DEG\ENaC family contribute to mechano-transduction, however the role of ASICs in sensory physiology is not well understood. Recent evidence indicates that ASICs mediate neuronal signaling due to tissue ischemia and inflammation. Under ischemic conditions, extracellular proton concentrations increase to levels sufficient for ASIC activation. Application of protons to skin causes pain in humans and this pain is blocked by prior application of ASIC antagonist (J Neurosci (2004) 24, 10974-10979). Painful sensation to acid likely originates from activation of ASIC channels that are expressed in the peripheral terminals of primary afferent nociceptors (Mol Pain (2005) 1, 35).

Sensory ASICs are assembled from homomeric and heteromeric combinations of at least five ASIC subunits (ASIC1a, ASIC1b, ASIC2a, ASIC2b, ASIC3). ASIC expression in small diameter sensory neurons may be particularly relevant to pain signaling as this population of sensory neurons is enriched in nociceptors. Here, the major isoforms of ASIC channels probably contain ASIC1a and ASIC3 homomers, and heteromeric combinations of ASIC1, ASIC2, and ASIC3. ASIC3 expression overlaps with other nociceptive markers supporting its role in pain signaling (Mol Pain (2005) 1, 35).

ASIC3 may be central to transmitting sensory information from skeletal muscle. Intense muscle activity leads to proton and lactic acid accumulation. Lactate potentiates the effects of protons on ASIC3(Nat Neurosci (2001) 4, 869-8701). The ASIC3 subunit is especially sensitive to small decreases in extracellular pH and more ASIC3 is found in muscle than in skin afferents (Mol Pain (2005) 1, 35). Block of ASIC function by ASIC antagonists or by genetic deletion of ASIC3 reduces pain-related behaviors to direct injection of protons into muscle (Pain (2003) 106, 229-239). Sensitivity to acid injection can be restored by viral expression of ASIC3 in sensory afferents of muscle but not skin of ASIC3 knockout mice (Pain (2007) 129, 102-112). Mechanical hypersensitivity to muscle inflammation is also diminished in ASIC3 knockout mice. This suggests that ASIC3 contributes to pain resulting from muscle inflammation.

ASIC may also play a sensory role in other tissues. During a heart attack, cardiac tissue becomes ischemic and lactic acid accumulates. Cardiac sensory neurons express high levels of ASIC3-containing channels. Modest decreases in pH are sufficient to evoke a sustained current in ASIC3-containing cardiac afferents. ASIC channels may also contribute to sensation from gastrointestinal tract. ASIC1, ASIC2, and ASIC3 knockout mice have altered sensitivity to mechanical stimulation in the gut (Gut (2005) 54, 1408-1415). These changes are accompanied by altered colonic function. Although ASIC belongs to a family of channels that includes invertebrate mechanosensitive channels, it is not clear how mechanical stimulation translates into ASIC activation.

In the brain, ASIC1 activation may mediate neuronal injury following a stroke.

The ASIC1 gene is the most highly expressed ASIC in the brain and is also the most permeable to calcium. The higher calcium permeability of ASIC1 may be an important factor in its contribution to neuronal injury. Psalmotoxin, a selective inhibitor of ASIC1 channels, or deletion of the ASIC1 gene decreases the size of brain damage in a stroke model. Interestingly, ASIC1 genetic deletion results in alteration of fear-related behaviors and hippocampal function and suggests that ASICs may also function in brain under non-pathological conditions.

Much less is known of the physiological function of ASIC2 and ASIC4 channels. ASIC4 is highly expressed in the pituitary, yet its function and endogenous activator are unknown. ASIC2 has two splice variants, ASIC2a and ASIC2b. Both variants have widespread tissue distribution, yet ASIC2 knockout animals show subtle phenotypes (J Physiol (2004) 558, 659-669). ASIC2 is expressed in sensory neurons that innervate cutaneous and visceral tissues and may contribute to mechanosensitivity in the gut (Gut (2005) 54, 1408-1415). ASIC2b and ASIC4 do not form proton-activated currents when expressed as homomers (although ASIC2b can form functional heteromers with other ASIC subunits).

The present invention is directed to novel compounds which are ASIC channel modulators, pharmaceutical compositions containing such compounds, and methods of using them as therapeutic agents.

### SUMMARY OF THE INVENTION

The present invention provides 2-aryl or heteroaryl indole derivatives which are

ASIC channel modulators, pharmaceutical compositions containing such compounds, and methods of using them as therapeutic agents.

### BRIEF DESCRIPTION OF THE DRAWINGS

FIG. 1. This figure shows dose dependent inhibition of human ASIC3 current with increasing concentrations of EXAMPLE 1 (closed circles) and EXAMPLE 4 (opened circles).

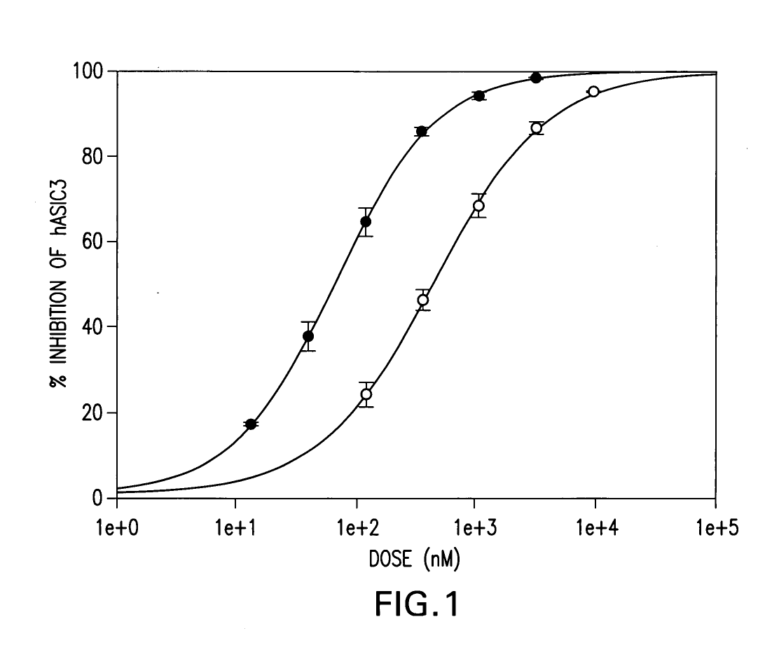

FIG. 2. This figure shows that EXAMPLE 1 inhibits proton-evoked current in HEK cells that express the human isoforms of ASIC3 (closed circles) and ASIC1 (open circles).

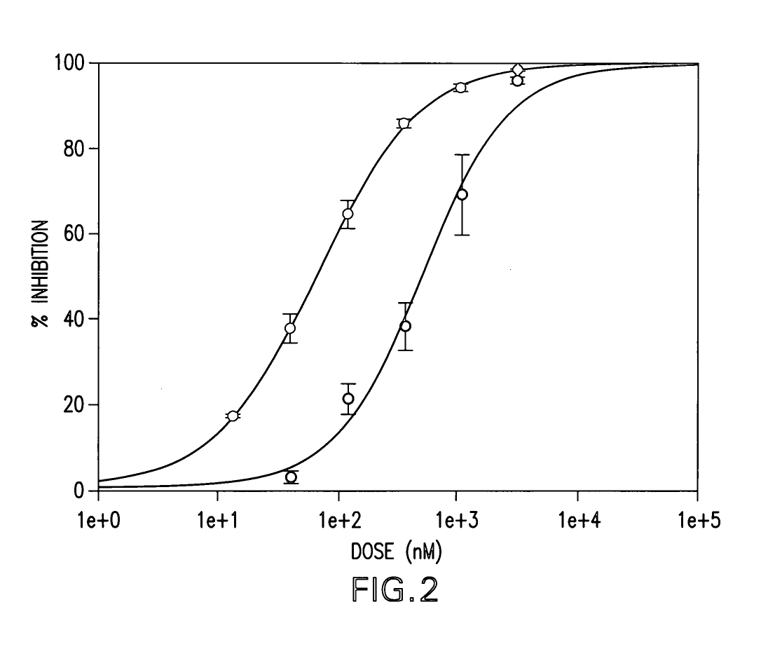

FIG. 3. This figure shows the inward current evoked by pH6.5 in the hASIC1 cell line. Pre-application of 300 nM EXAMPLE 1 inhibits a large fraction of this current.

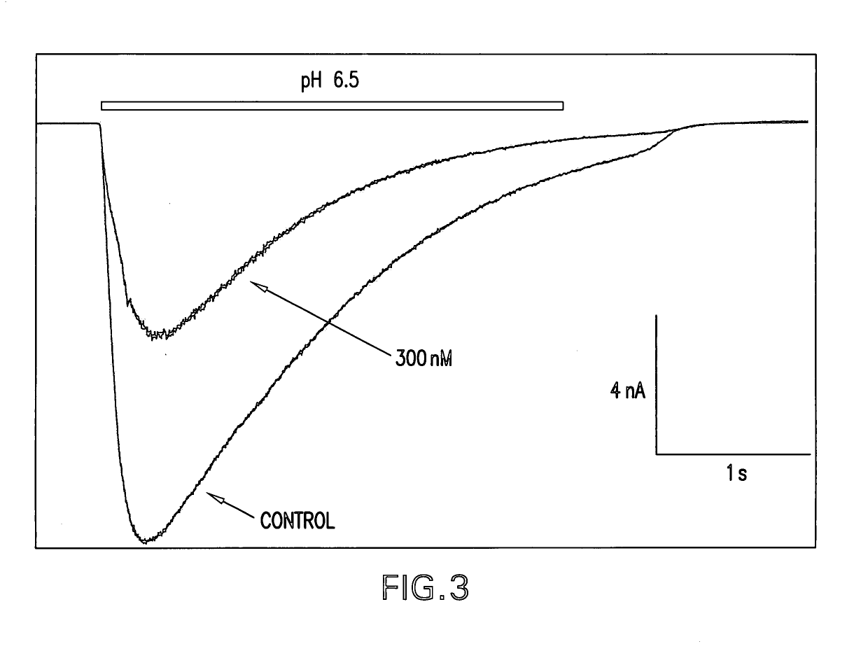

FIG. 4. This figure shows the inward current evoked by pH5.5 in the hASIC3 cell line. Pre-application of 300 nM EXAMPLE 1 inhibits the majority of the proton-evoked current.

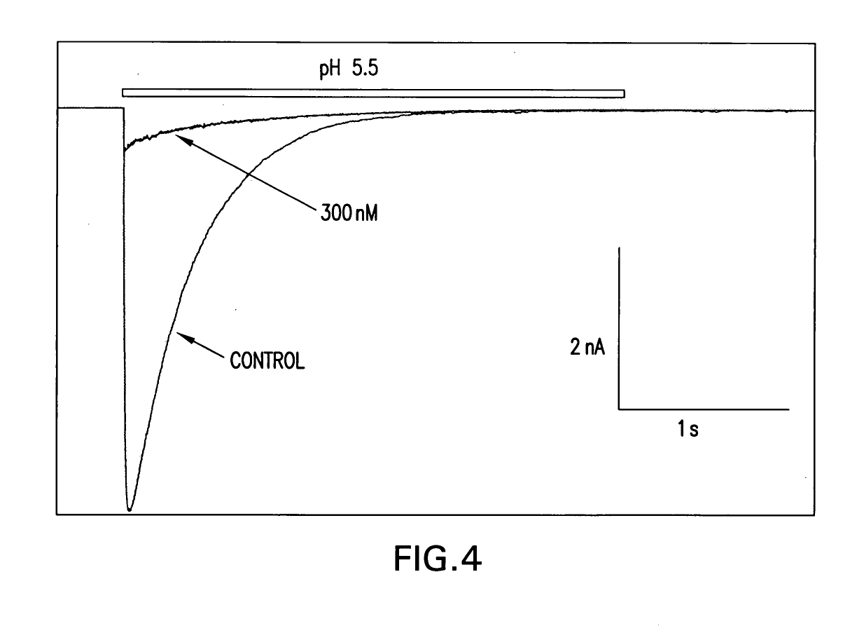

FIG. 5. This figure shows the inward current evoked by pH5.5 in the hASIC3 cell line. Pre-application of 10 μM EXAMPLE 2 did not block the proton-evoked current.

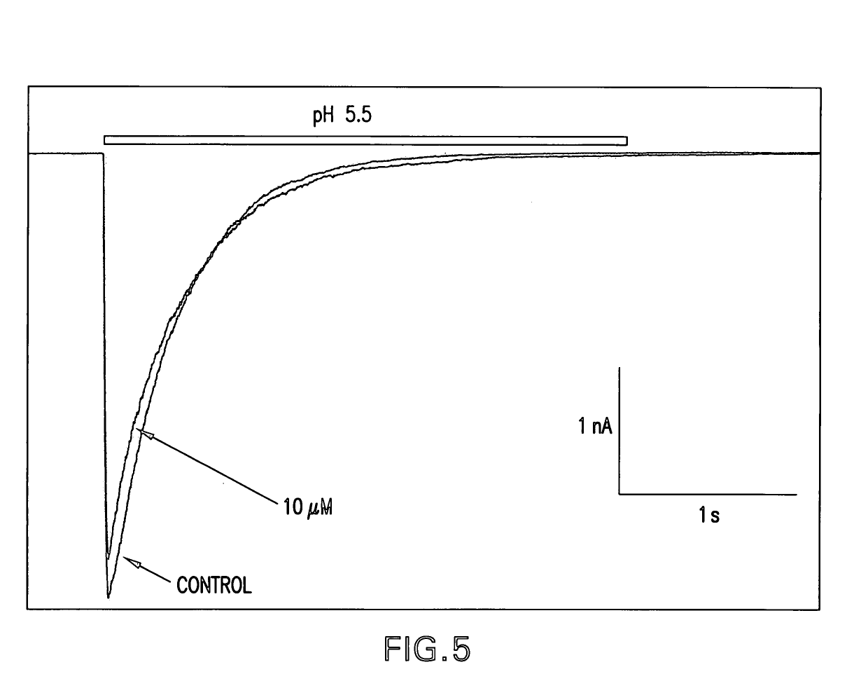

FIG. 6. This figure shows the inward current evoked by pH5.5 in the hASIC3 cell line. Pre-application of 10 μM EXAMPLE 3 blocked the proton-evoked current.

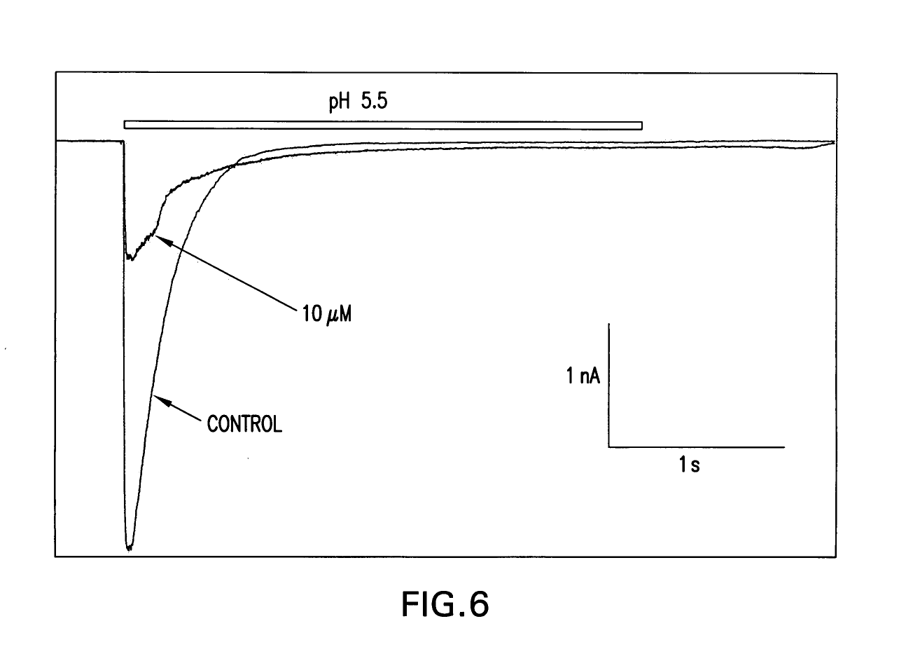

FIG. 7. This figure shows the inward current evoked by pH5.5 in the hASIC3 cell line. Pre-application of 300 nM EXAMPLE 4 inhibited the proton-evoked current.

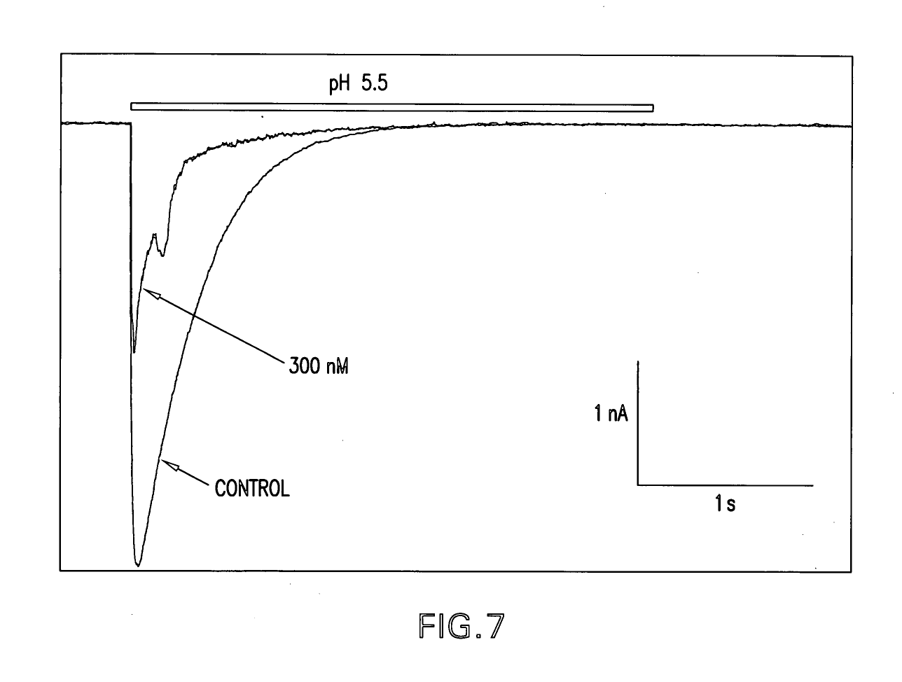

### DETAILED DESCRIPTION OF THE INVENTION

The invention encompasses a genus compound according to Formula I or Formula

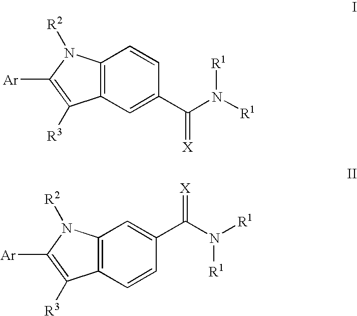

or a pharmaceutically acceptable salt of a compound of Formula I or Formula II, wherein:

### X=NH or H,H

each R¹ is independently selected from the group consisting of: H, —OR², —CO₂R², —CON(R²)₂, C₁₋₄alkyl, C₁₋₄alkyloxy, phenyl, benzyl, —CN and —COR²; or the two R¹ groups can be joined together with the nitrogen atom to which they are attached to form a 5- or 6-membered monocyclic ring, optionally containing a heteroatom selected from O, S or N;
each R² is independently selected from the group consisting of: H, C₁₋₈ alkyl, C₃₋₈ cycloalkyl and phenyl;
R³ is selected from the group consisting of: H, C₁₋₈alkyl, C₂₋₈alkenyl, C₂₋₈alkynyl, C₃₋₈cycloalkyl, halogen, C₁₋₄alkyloxy, —CN, —NO₂, —N(R⁵)₂, —NR⁵COR⁴, —CO₂R⁴, —COR⁴, —OH, —SR⁴, —SOR⁴, —SO₂R⁴, —SO₂NHR⁵, —NONHR⁵ and phenyl, wherein said C₁₋₈alkyl is optionally substituted with 1 to 3 substituents independently selected from the group consisting of: halogen, OR⁴, nitro, cyano, C₁₋₄alkyloxy, COR⁴, SO₂R⁴ and CO₂R⁴; and
Ar is aryl or heteroaryl, each optionally substituted with 1 to 5 R^{a} groups, wherein each R^{a} is independently selected from the group consisting of: halogen, —OR⁴, —S—R⁴, —CO₂R⁴, —COR⁴, —NO₂, —N(R⁵)₂, C₁₋₄alkyl, C₁₋₄haloalkyl, C₁₋₄hydroxyalkyl, aryl, heteroaryl, N(R⁵)₂—CH₂—, N(R⁵)₂—SO₂—, —CN, CN—CH₂—, —CF₃, N(R⁵)₂—C(O)—CH₂—, R⁴—SO₂—N(H)—CH(R³)—CH₂—, CH₃—C(O)—CH═CH—, heterocyclyl-C₁₋₄-alkylenyl-O—, R⁴—O—C(O)—NH—CH(R³)—CH₂—, R⁵—N(H)—(CH₂)₃—N(H)— and phenyl substituted with 1 to 5 substituents independently selected from the group consisting of: halo, C₁₋₄alkyl and C₁₋₄haloalkyl;
each R⁴ is independently selected from the group consisting of: H, C₁₋₈ alkyl, C₁₋₄haloalkyl, C₃₋₈ cycloalkyl, phenyl and benzyl; and
each R⁵ is independently selected from the group consisting of: H, C₁₋₈ alkyl, C₃₋₈ cycloalkyl, phenyl, benzyl, OR² or two R⁵ groups can be joined together with the nitrogen atom to which thy are attached to form a six membered monocyclic ring.

Within the genus, the invention encompasses a sub-genus of compounds having Formula I.

Within the sub-genus, the invention encompasses a first class of compounds of Formula I wherein R² is H.

Also within the sub-genus, the invention encompasses a second class of compounds of Formula I wherein X is NH.

Also within the sub-genus, the invention encompasses a third class of compounds of Formula I wherein X is H,H.

Also within the sub-genus, the invention encompasses a fourth class of compounds of Formula I wherein Ar is phenyl, optionally substituted with 1 to 5 R^{a} groups.

Also within the sub-genus, the invention encompasses a fifth class of compounds of Formula I wherein Ar is pyridyl, optionally substituted with 1 to 5 R^{a} groups.

Also within the sub-genus, the invention encompasses a sixth class of compounds of Formula Ia

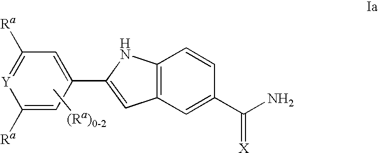

or a pharmaceutically acceptable salt thereof, wherein

### X=NH or H,H;

### Y is CH or N;

each R^{a} is independently selected from the group consisting of: halogen, —OR, —S—R⁴, —CO₂R⁴, —COR⁴, —NO₂, —N(R⁵)₂, C₁₋₄alkyl, C₁₋₄haloalkyl, C₁₋₄hydroxyalkyl, aryl, heteroaryl, N(R⁵)₂—CH₂—, N(R⁵)₂—SO₂—, —CN, CN—CH₂—, —CF₃, N(R⁵)₂—C(O)—CH₂—, R⁴—SO₂—N(H)—CH(R³)—CH₂—, CH₃—C(O)—CH═CH—, heterocyclyl-C₁₋₄-alkylenyl-O—, R⁴—O—C(O)—NH—CH(R³)—CH₂—, R⁵—N(H)—(CH₂)₃—N(H)— and phenyl substituted with 1 to 5 substituents independently selected from the group consisting of: halo, C₁₋₄alkyl and C₁₋₄haloalkyl;
each R⁴ is independently selected from the group consisting of: H, C₁₋₈ alkyl, C₁₋₄haloalkyl, C₃₋₈ cycloalkyl, phenyl and benzyl; and
each R⁵ is independently selected from the group consisting of: H, C₁₋₈ alkyl, C₃₋₈ cycloalkyl, phenyl, benzyl, OR² or two R⁵ groups can be joined together with the nitrogen atom to which thy are attached to form a six membered monocyclic ring.

Within the sixth class, the invention encompasses a sub-class of compounds of Formula Ia wherein each R^{a} is independently selected from the group consisting of: HO—CH₂—, F, Cl, Br, NH₂, CH₃—O—, —CN, —CH₃, phenyl, phenylamino, benzylamino, formyl and 1-piperidinylmethyl. Within this sub-class, the invention encompasses compounds of Formula Ia wherein X is NH. Also within the sub-class, the invention encompasses compounds of Formula Ia wherein X is H,H.

The invention also encompasses a compound selected from Examples 1 to 31, 35 to 42, 44 to 156, 158 to 210, 212 to 222, 224 to 239, 241 to 249, 251 to 256 and 258 to 261, or a pharmaceutically acceptable salt of any of these examples.

The invention also encompasses a pharmaceutical composition comprising a compound of Formula I in combination with a pharmaceutically acceptable carrier.

The invention also encompasses a method for treating pain in a mammalian patient in need thereof comprising administering to the patient a therapeutically effective amount of a compound of Formula I or Formula II.

The invention also encompasses a method for treating a disease or condition selected from the group consisting of: (1) chronic, visceral, inflammatory and neuropathic pain syndromes; (2) pain resulting from traumatic nerve injury, nerve compression or entrapment, postherpetic neuralgia, trigeminal neuralgia, and diabetic neuropathy; (3) chronic lower back pain, phantom limb pain, chronic pelvic pain, neuroma pain, complex regional pain syndrome, chronic arthritic pain and related neuralgias, and (4) pain associated with cancer, chemotherapy, HIV and HIV treatment-induced neuropathy, in a mammalian patient in need thereof comprising administering to the patient a therapeutically effective amount of a compound of Formula I or Formula II.

The invention also encompasses a method for treating a disease or condition selected from the group consisting of: stroke, anxiety, bone cancer pain, bone disorders with increased osteoclastic bone resorption, ischemic pain, sickle cell anemia, anemia pain, intermittent claudication, gastric mobility disorders, irritable bowel syndrome or disease, inflammatory bowel syndrome or disease and satiety-obesity, in a mammalian patient in need thereof comprising administering to the patient a therapeutically effective amount of a compound of Formula I or Formula II. In another embodiment, the disease or condition is bone disorders with increased osteoclastic bone resorption selected from the group consisting of: metastatic bone diseases, Paget's disease of bone, osteoporosis, fibrous dysplasia and osteogenesis imperfecta.

### DEFINITIONS

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Haloalkyl” means alkyl as defined above wherein one or more hydrogen atoms have been replaced by halogen atoms.

“Hydroxyalkyl” means alkyl as defined above wherein one or more hydrogen atoms have been replaced by hydroxy atoms.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, having the indicated number of carbon atoms, or a fused analog thereof. A “fused analog” of cycloalkyl means mono- or bicyclic saturated carbocyclic rings fused to one or more aryl or heteroaryl groups in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like.

“Alkoxy” means alkoxy groups of a straight or branched having the indicated number of carbon atoms. C₁₋₆alkoxy, for example, includes methoxy, ethoxy, propoxy, isopropoxy, and the like.

“Cycloalkoxy” means cycloalkyl as defined above bonded to an oxygen atom, such as cyclopropyloxy.

“Fluoroalkoxy” means alkoxy as defined above wherein one or more hydrogen atoms have been replaced by fluoro atoms.

“Aryl” means mono- or bicyclic aromatic rings containing only carbon atoms or a fused analog thereof. A “fused analog” means mono- or bicyclic aromatic rings containing only carbon atoms fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one heteroatom selected from N, O and S, with each ring containing 5 to 6 atoms, or a fused analog thereof. A “fused analog” means a mono- or bicyclic aromatic ring containing at least one heteroatom selected from N, O and S, with each ring containing 5 to 6 atoms, fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Heterocyclyl” means mono- or bicyclic saturated rings or partially unsaturated monocyclic rings that are not aromatic containing at least one heteroatom selected from N, S and O, each of said rings having from 3 to 10 atoms in which the point of attachment may be carbon or nitrogen, optionally substituted with oxo, or a fused analog thereof. A “fused analog” of heterocyclyl means mono- or bicyclic saturated rings or partially unsaturated monocyclic rings that are not aromatic, containing at least one heteroatom selected from N, S and O, each of said rings having from 3 to 10 atoms, fused to one or more aryl or heteroaryl groups in which the point of attachment is on the non-aromatic portion, which may be carbon or nitrogen. Examples of “heterocyclyl” and fused analogs thereof include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” and “halo” includes fluorine, chlorine, bromine and iodine.

### Optical Isomers—Diastereomers —Geometric Isomers—Tautomers

Compounds of Formula I or Formula II contain one or more asymmetric centers and can thus occur as racemates and racemic mixtures, single enantiomers, diastereomeric mixtures and individual diastereomers. The present invention is meant to comprehend all such isomeric forms of the compounds of Formula I or Formula II.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I or Formula II.

Compounds of the Formula I or Formula II may be separated into diastereoisomeric pairs of enantiomers by, for example, fractional crystallization from a suitable solvent, for example MeOH or EtOAc or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I or Formula II may be obtained by stereospecific synthesis using optically pure starting materials or reagents of known configuration.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethyl-aminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methyl-glucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I or Formula II are meant to also include the pharmaceutically acceptable salts.

### Utilities

The compounds of the invention are ASIC3 inhibitors and as such are useful for treating diseases or conditions mediated by ASIC channels. Thus, the compounds of the invention are useful in the treatment of the disorders that follow.

The compounds of the invention are useful as analgesics. For example they are useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

The compounds of the invention are useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that 25 precipitated them. Neuropathic pain syndromes include: diabetic neuropathy; sciatica; non-specific lower back pain; multiple sclerosis pain; fibromyalgia; HIV related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), 35 increased sensitivity to touch (hyperesthesias), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The present compounds and compositions are useful for the treatment of chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, and pain associated with cancer, chemotherapy, HIV and HIV treatment-induced neuropathy. Compounds of this invention may also be utilized as local anesthetics.

Compounds of this invention are useful for the treatment of irritable bowel syndrome and related disorders, as well as Crohns disease.

The compounds of the invention are also useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome pigeon fancier's disease, farmer's lung, CORD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Becheto's syndrome, polymyositis, gingivitis, myocardial ischemia, pyrexia, systemic lupus erythematosus, polymyositis, tendinitis, bursitis, and Sjogren's syndrome.

The compounds of the invention are also useful in the treatment of immunological diseases such as autoimmune diseases, immunological deficiency diseases or organ transplantation. The compounds of the invention are also effective in increasing the latency of HIV infection.

The compounds of the invention are also useful in the treatment of diseases of abnormal platelet function (e.g. occlusive vascular diseases).

The compounds of the invention are also useful for the preparation of a drug with diuretic action.

The compounds of the invention are also useful in the treatment of impotence or erectile dysfunction.

The compounds of the invention are also useful in the treatment of bone disease characterized by abnormal bone metabolism or resorption such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis. In a further aspect compounds of the invention may be useful in inhibiting bone resorption and/or promoting bone generation.

The compounds of the invention are also useful for attenuating the hemodynamic side effects of NSAIDs.

The compounds of the invention are also useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of the invention are also useful in the treatment of neurodegenerative diseases and neurodegeneration such as dementia, particularly degenerative dementia (including senile dementia, Alzheimer's disease, Pick's disease, Huntingdon's chores, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of the present invention are useful for treating a variety of neurological and psychiatric disorders, including one or more of the following conditions or diseases: mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder, mood disorders due to a general medical condition, and substance-induced mood disorders; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia); obesity, bulimia nervosa, compulsive eating disorders, eating disorders associated with excessive food intake and complications associated therewith; conduct disorder; muscular spasms and disorders associated with muscular spasticity or weakness including tremors; urinary incontinence; amyotrophic lateral sclerosis; neuronal damage including ocular damage, retinopathy or macular degeneration of the eye, hearing loss or tinnitus; emesis, brain edema and sleep disorders including insomnia and narcolepsy.

The compounds of Formula I or Formula II are also useful in the treatment of neuroprotection and in the treatment of neurodegeneration following stroke, cardiac arrest, pulmonary bypass, traumatic brain injury, spinal cord injury or the like. The compounds of the present invention have utility in treating, ameliorating or controlling stroke and the neurological injuries caused by stroke. As used herein, the term “stroke” refers to a clinical event involving impairment of cerebral circulation, resulting in neurological injury. Typically, stroke is manifest by the abrupt onset of a focal neurological deficit. Stroke results from a rupture or obstruction (as by a thrombus or embolus) of an artery of the brain.

The compounds of the invention are also useful in the treatment of tinnitus.

The compounds of the invention are also useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of the invention are also useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of the invention are also useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The compounds of the invention are also useful for treating or preventing a neoplasia in a subject in need of such treatment or prevention. The term “treatment” includes partial or total inhibition of the neoplasia growth, spreading or metastasis, as well as partial or total destruction of the neoplastic cells. The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia. The term “subject” for purposes of treatment includes any human or mammal subject who has any one of the known neoplasias, and preferably is a human subject. For methods of prevention, the subject is any human or animal subject, and preferably is a human subject who is at risk for obtaining a neoplasia. The subject may be at risk due to exposure to carcinogenic agents, being genetically predisposed to have the neoplasia, and the like. The term “neoplasia” includes both benign and cancerous tumors, growths and polyps. Thus, the compounds of the invention are useful for treating or preventing benign tumors, growths and polyps including squamous cell papilloma, basal cell tumor, transitional cell papilloma, adenoma, gastrinoma, cholangiocellular adenoma, hepatocellular adenoma, renal tubular adenoma, oncocytoma, glomus tumor, melanocytic nevus, fibroma, myxoma, lipoma, leiomyoma, rhabdomyoma, benign teratoma, hemangioma, osteoma, chondroma and meningioma. The compounds of the invention are also useful for treating or preventing cancerous tumors, growths and polyps including squamous cell carcinoma, basal cell carcinoma, transitional cell carcinoma, adenocarcinoma, malignant gastrinoma, cholangiocelleular carcinoma, hepatocellular carcinoma, renal cell carcinoma, malignant melanoma, fibrosarcoma, myxosarcoma, liposarcoma, leimyosarcoma, rhabdomyosarcoma, malignant teratoma, hemangiosarcoma, Kaposi sarcoma, lymphangiosarcoma, ostreosarcoma, chondrosarcoma, malignant meningioma, non-Hodgkin lymphoma, Hodgkin lymphoma and leukemia. For purposes of this specification, “neoplasia” includes brain cancer, bone cancer, epithelial cell-derived neoplasia (epithelial carcinoma), basal cell carcinoma, adenocarcinoma, gastrointestinal cancer such as lip cancer, mouth cancer, esophogeal cancer, small bowel cancer and stomach cancer, colon cancer, rectal cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that affect epithelial, mesenchymal or blood cells throughout the body. The compounds of the invention are useful for treating or preventing any of the aforementioned cancers. The compounds of the invention are useful for treating or preventing benign and cancerous tumors, growths and polyps of the following cell types: squamous epithelium, basal cells, transitional epithelium, glandular epithelium, G cells, bile ducts epithelium, hepatocytes, tubules epithelium, melanocytes, fibrous connective tissue, cardiac skeleton, adipose tissue, smooth muscle, skeletal muscle, germ cells, blood vessels, lymphatic vessels, bone, cartilage, meninges, lymphoid cells and hematopoietic cells. The compounds can be used to treat subjects having adenomatous polyps, including those with familial adenomatous polyposis (FAP). Additionally, the compounds can be used to prevent polyps from forming in patients at risk of FAR Preferably, the compounds of the invention are useful for treating or preventing the following cancers: colorectal, esophagus stomach, breast, head and neck, skin, lung, liver, gall bladder, pancreas, bladder, endometrium cervix, prostate, thyroid and brain.

Preferably, the compounds of the invention are useful for treating bone cancer pain, stroke, anxiety, bone disorders with increased osteoclastic bone resorption, such as metastatic bone diseases, Paget's disease of bone, osteoporosis, fibrous dysplasia and osteogenesis imperfecta, ischemic pains, sickle cell anemia, anemia pain, intermittent claudication, gastric mobility disorders, irritable bowel syndrome or disease, inflammatory bowel syndrome or disease and satiety-obesity.

It is to be understood that reference to treatment includes both treatment of established symptoms and prophylactic treatment, unless explicitly stated otherwise.

### Dose Ranges

The magnitude of prophylactic or therapeutic dose of a compound of Formula I or Formula II will, of course, vary with the nature and severity of the condition to be treated, and with the particular compound of Formula I or Formula II used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I or Formula II per kg of body weight per day.

In the case where an oral composition is employed, a suitable dosage range is, e.g. from about 0.01 mg to about 100 mg of a compound of Formulas I or I a per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I or Formula II per kg of body weight per day.

### Pharmaceutical Compositions

Another aspect of the present invention provides pharmaceutical compositions which comprises a compound of Formula I or Formula II and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I or Formula II, additional active ingredient(s), and pharmaceutically acceptable excipients.

Any suitable route of administration may be employed for providing a mammal, especially a human with an effective dosage of a compound of the present invention. For example, oral, sublingual, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I or Formula II as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I or Formula II in suitable propellants, such as fluorocarbons or hydrocarbons and dry powder inhalation (DPI) aerosol, which may be formulated as a dry powder of a compound of Formula I or Formula II with or without additional excipients.

Suitable topical formulations of a compound of Formula I or Formula II include transdermal devices, aerosols, creams, ointments, lotions, dusting powders, and the like.

In practical use, the compounds of Formula I or Formula II can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques:

In addition to the common dosage forms set out above, the compounds of Formula I or Formula II may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### Combination Therapy

Compounds of Formula I or Formula II may be used in combination with other drugs that are used in the treatment/prevention/suppression or amelioration of the diseases or conditions for which compounds of Formula I or Formula II are useful. Such other drugs may be administered, by a route and in an amount commonly used therefor, contemporaneously or sequentially with a compound of Formula I or Formula II. When a compound of Formula I or Formula II is used contemporaneously with one or more other drugs, a pharmaceutical composition containing such other drugs in addition to the compound of Formula I or Formula II is preferred. Accordingly, the pharmaceutical compositions of the present invention include those that also contain one or more other active ingredients, in addition to a compound of Formula I or Formula II. Examples of other active ingredients that may be combined with a compound of Formula I or Formula II, either administered separately or in the same pharmaceutical compositions, include, but are not limited to: COX-2 inhibitors, such as celecoxib, rofecoxib, etoricoxib, valdecoxib or parecoxib; 5-lipoxygenase inhibitors; NSAIDs, such as diclofenac, indomethacin, nabumetone or ibuprofen; leukotriene receptor antagonists; DMARDs such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT agonists, such as triptans, for example sumatriptan, naratriptan, zolmitriptan, eletriptan, frovatriptan, almotriptan or rizatriptan; EP1 receptor ligands; EP2 receptor ligands; EP3 receptor ligands; EP4 receptor ligands; EP1 antagonists; EP2 antagonists; EP3 antagonists and EP4 antagonists. When the compounds are used in combination with other therapeutic agents, the compounds may be administered either sequentially or simultaneously by any convenient route.

Compounds of the invention may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

Compounds of the invention may be employed in combination with anti-Alzheimer's agents; beta-secretase inhibitors; gamma-secretase inhibitors; HMG-CoA reductase inhibitors; NSAID's including ibuprofen; vitamin E; anti-amyloid antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H₃ antagonists; AMPA agonists; PDE IV inhibitors; GABA_{A} inverse agonists; or neuronal nicotinic agonists.

Compounds of the invention may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the subject compound may be administered in conjunction with the use of physical methods such as with light therapy or electrical stimulation.

Compounds of the invention may be employed in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexol)hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

Compounds of the invention may be employed in combination with acetophenazine, alentemol, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, risperidone, sulpiride, tetrabenazine, trihexyphenidyl, thioridazine, thiothixene or trifluoperazine.

Compounds of the invention may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form.

Compounds of the invention may be employed in combination with an anoretic agent such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; selective serotonin reuptake inhibitor (SSRI); halogenated amphetamine derivatives, including chlorphentermine, cloforex, clortermine, dexfenfluramine, fenfluramine, picilorex and sibutramine; and pharmaceutically acceptble salts thereof.

Compounds of the invention may be employed in combination with an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the subject compound may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

The invention thus provides, in a further aspect, a combination comprising a compound of Formula I or Formula II or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

The weight ratio of the compound of the Formula I or Formula II to the second active ingredient may be varied and will depend upon the effective dose of each ingredient. Generally, an effective dose of each will be used. Thus, for example, when a compound of Formula I or Formula II is combined with an NSAID the weight ratio of the compound of Formula I or Formula II to the NSAID will generally range from about 1000:1 to about 1:1000, preferably about 200:1 to about 1:200. Combinations of a compound of Formula I or Formula II and other active ingredients will generally also be within the aforementioned range, but in each case, an effective dose of each active ingredient should be used.

### Biological Methods

### In Vitro Assay for ASIC Activity

Cell Preparation: Human embryonic kidney cells (HEK293) stably expressing the human ASIC ion channel, are grown to 70-90% confluence in T75 cell culture flasks. Cells are lifted by removing the growth media and incubating with 3 ml of warmed (37° C.) 50/50 Trypsin/Dulbecco's Solution (GIBCO, USA) for 4-6 minutes. 7 ml of pH7.4_HBS (see Solutions and Drugs) is then added to block Trypsin activity. The cell suspension is placed into a 10 ml centrifuge tube and centrifuged for 1 minute @ 150×G. After centrifugation, the supernatant is aspirated and the cell pellet is re-suspended in 2-5 ml of pH7.4_HBS for a final cell density of ˜0.6×10⁶ cells/ml. For whole-cell patch clamp, approximately 1000 cells are placed in a recording chamber containing standard bath solution for electrophysiological recording.

Solutions and Drugs: The intracellular solution contains (mM): 119 K-gluconate, 15 KCl, 3.2 MgCl₂, 5 EGTA, 5 HEPES, 5 K2ATP, pH 7.3. The standard bath solution (pH7.4_HBS) contains (mM): 150 NaCl, 5 KCl, 2 CaCl₂, 1 MgCl₂, 10 HEPES, 12 Dextrose, pH 7.4. The acid-activation bath solution (pH5.5_MBS) contains (mM): 150 NaCl, 5 KCl, 2 CaCl₂, 1 MgCl₂, 10 MES, 12 Dextrose, pH 5.5. Test compounds were prepared as 2 or 10 mM stock solutions in DMSO and diluted in either pH7.4_HBS or pH5.5_MBS to final concentration.

Electrophysiological recording: Patch clamp recording pipettes were pulled from borosilicate glass and typically had 2-4 MOhm resistances. Whole cell currents were recorded with a patch clamp amplifier (Multiclamp 700B, Axon Instruments Inc). Holding and test potential was −60 mV. Control and compound solutions perfused the vicinity of the cell with flow controlled by computer-operated solenoid valves. 10-90% solution exchange typically took <20 ms. After establishment of whole cell configuration, cells were perfused with pH7.4_HBS. Baseline ASIC current was determined by application of pH5.5_MBS at two minute intervals. After a stable ASIC current was determined, inhibition of acid evoked current was determined by multiple applications of increasing concentrations (3-fold dilution) of compound. Inhibition of current was expressed as percent inhibition vs. baseline current. A minimum of three independent determinations at each concentration of compound was used for each data point. IC50s were determined using the Hill Equation.

### Examples 1 to 261 were tested in the above assay. Examples 1 to 31, 35 to 42, 44 to 156, 158 to 210, 212 to 222, 224 to 239, 241 to 249, 251 to 256 and 258 to 261 showed levels of inhibition of greater than 20% at 10 uM.

### Abbreviations Used

The following abbreviations have the meanings indicated, unless stated otherwise in the specification: Ac=acetyl; Boc=t-butoxycarbonyl; Cat.=catalyst; DCM=dichloromethane; DMF=dimethylformamide; DMSO=dimethyl sulfoxide; EDCI=1-(3-dimethylaminopropyl)-3-ethylcarbodiimide; Et=ethyl; EtOAc=ethyl acetate; EtOH=ethanol; HOBT=1-hydroxybenzo-triazole; LAH=lithium aluminum hydride; LDA=lithium diisopropylamide; Me=methyl; MeOH=methanol; NBS=N-bromosuccinimide; NMP=N-methylpyrrolidinone; NMO=N-methylmorpholino N-oxide; Ph=phenyl; Rt=room temperature; TEA=triethylamine; TFA=trifluoroacetic acid; THF=tetrahydrofuran.

### Methods of Synthesis

Compounds of Formula I or Formula II may be prepared by the general procedure depicted in Scheme 1.

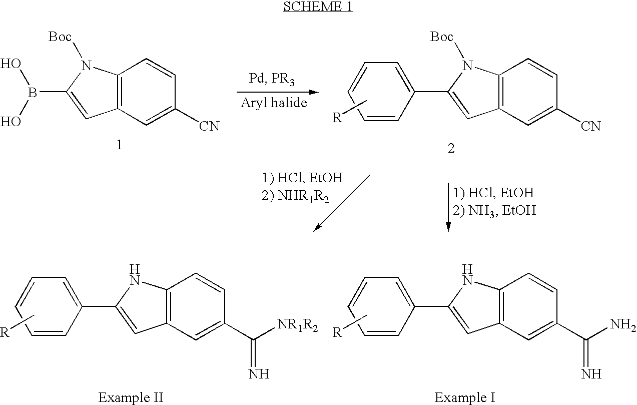

Compound 1 is commercially available or may be prepared from commercially available reagents using conventional chemical reactions well known in the art. Intermediate 2 may be prepared via cross-coupling of 1 with an appropriate aryl halide using a metal catalyst such as palladium with a tri-substituted phosphine, in an appropriate solvent, like dimethyl sulfoxide. The Boc group of 2 may be removed and the nitrile converted to an imidate HCl salt which can be treated with an ammonia source in an alcohol solvent to provide amidine example I. Alternatively, the imidate salt of compound 2 may be treated with a primary or secondary amine to provide a substituted amidine example II.

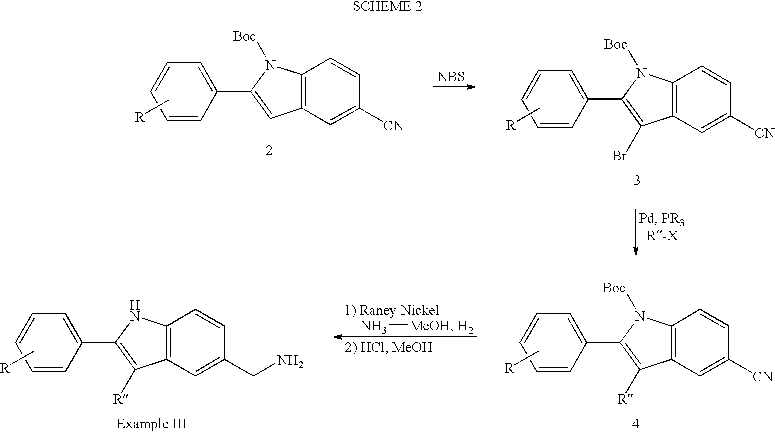

Scheme 2 describes the general synthesis of indoles with substitution at the 3-position. Bromination of compound 2 with N-bromosuccinimide in a solvent like acetonitrile affords compound 3. Compound 3 may be converted with a transition metal like palladium or nickel and the appropriate reagent (R″=aryl or alkyl, X=B(OH)₂, SH, NH₂, OH, Zn) into compound 4. Reduction of 4 using a metal catalyst such Raney Nickel in an alcohol solvent under an atmosphere of hydrogen followed by removal of the Boc protecting using an acid like HCl affords example III.

Alternatively, the compounds could be prepared as shown in Scheme 3. Reduction of commercially available 5-cyano indole followed by protection of the amine and indole nitrogen with a Boc group affords compound 5. Metallation of 5 using a strong base like LDA in a solvent like THF and quenching with a borate like triisopropyl borate affords compound 6. Subsequent cross-coupling and Boc removal as described in Scheme 1 affords example IV.

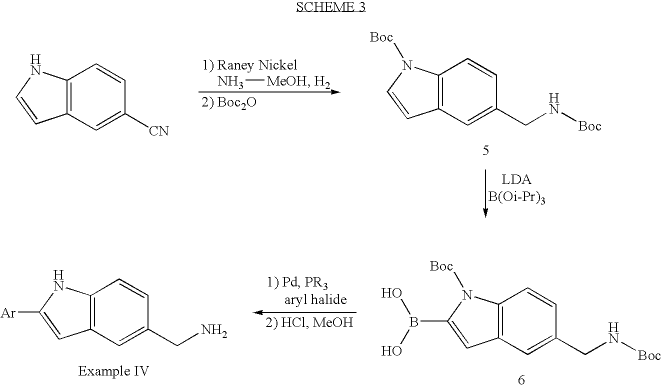

The following examples are provided to illustrate the invention and are not to be construed as limiting the scope of the invention in any manner.

### Example 1

### 2-(3,5-Dichlorophenyl)-1H-indole-5-carboximidamide

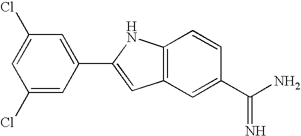

A mixture of 1-Boc-5-cyanoindole-2-boronic acid (4.0 g, 13.98 mmol), 1,3-dichloro-5-iodobenzene (4.58 g, 16.78 mmol), dicyclohexylamine (7.6 g, 41.90 mmol) and [1,1′-bis(diphenylphosphino)ferrocene]palladium(II) dichloride (0.57 g, 0.70 mmol) in THF (30 mL) was purged with nitrogen and was heated to 60° C. in a pressure vessel for 3 hours. The reaction mixture was cooled to room temperature, and was poured in a mixed solvent of EtOAc (50 mL) and hexanes (200 mL). The suspension was stirred vigorously at room temperature for 15 minutes, and was filtered to remove the solid residue. The filtrate was concentrated and the crude residue was purified by silica gel chromatography with 0-10% EtOAc in hexanes to provide tent-butyl 5-cyano-2-(3,5-dichlorophenyl)-1H-indole-1carboxylate that gave a mass ion (ES⁺) of 387 for M+H⁺ (³⁵Cl) and proton NMR spectra consistent with theory.

A solution of tert-butyl 5-cyano-2-(3,5-dichlorophenyl)-1H-indole-1-carboxylate (0.16 g, 0.56 mmol) in anhydrous EtOH (50 mL) was saturated with anhydrous HCl gas at room temperature in a pressure vessel, and heated at 50° C. for 6 hours. The solution was cooled to room temperature, and was concentrated. A suspension of the crude in anhydrous EtOH (50 mL) was saturated with anhydrous ammonia gas at room temperature in a pressure vessel, and was heated to 50° C. for 16 hours. After cooling to room temperature, the solution was concentrated and the residue was subjected to reverse phase liquid chromatography to provide 2-(3,5-dichlorophenyl)-1H-indole-5-carboximidamide trifluoroacetate that gave a mass ion (ES+) of 304 for M+H⁺ (³⁵Cl) and proton NMR spectra consistent with theory. ¹H NMR (400 MHz, DMSO-d₆) δ 12.26 (s, 1H), 9.18 (s, 2H), 8.79 (s, 2H), 8.16 (s, 1H), 8.04 (d, J=1.74 Hz, 2H), 7.62-7.57 (m, 3H), 7.39 (d, J=1.84 Hz, 1H).

Compounds in Tables 1-5 having a basic group or acidic group are depicted as the free base or acid. Depending on the reaction and purification conditions, various compounds having a basic group were isolated in either the free base form, or as a salt (such as HCl salt), or in both free base and salt forms. Various compounds having an acid group were isolated in either the acid form, or as a salt (such as Na salt), or in both acid and salt forms.

The following compounds were prepared according to the general procedure provided in Example 1. The starting materials are either commercially available or may be prepared from commercially available reagents using conventional reactions well known in the art.

TABLE 1
2-Substituted 1H-indole-5-carboximidamides

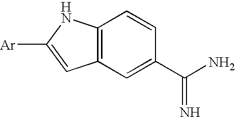

| | | MASS |
|---|---|---|
| | | SPEC |
| EXAMPLE | ARYL = | (M + H)⁺ |
| 2 | 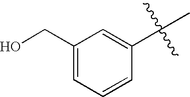 | 266.3 |
| 3 | 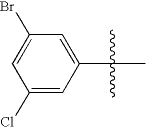 | 349.9 |
| 4 | 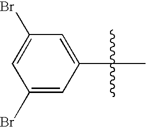 | 393.8 |
| 5 | 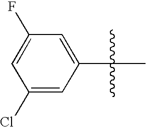 | 290.0 |
| 6 | 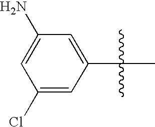 | 287.2 |
| 7 | 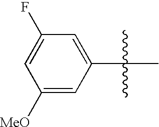 | 284.2 |
| 8 | 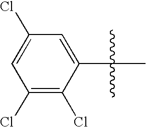 | 340.1 |
| 9 | 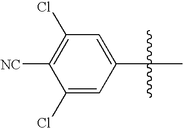 | 331.1 |
| 10 | 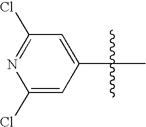 | 307.1 |
| 11 | 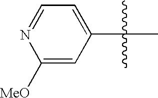 | 267.2 |
| 12 | 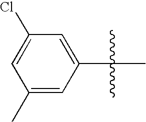 | 286.2 |
| 13 | 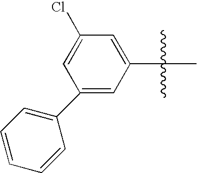 | 347.1 |
| 14 | 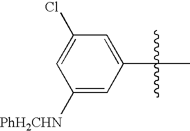 | 377.2 |
| 15 | 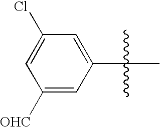 | 298.2 |
| 16 | 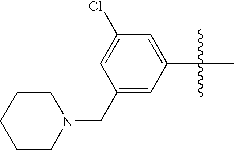 | 367.2 |
| 17 | 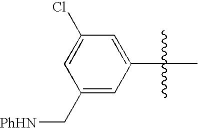 | 375.1 |

The following compounds were prepared according to the general procedure provided in Example 1 starting with 1-Boc-6-cyanoindole. The starting materials are either commercially available or may be prepared from commercially available reagents using conventional reactions well known in the art.

TABLE 2
2-Substituted lH-indole-6-carboximidamides

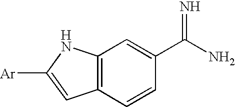

| | | MASS |
|---|---|---|
| | | SPEC |
| EXAMPLE | ARYL = | (M + H)⁺ |
| 18 | 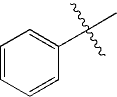 | 236.6 |
| 19 | 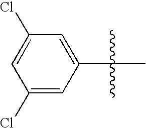 | 306.1 |
| 20 | 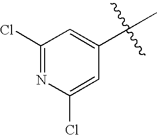 | 307.1 |

The following compounds were prepared according to the general procedure provided in Example 1. The starting materials are either commercially available or may be prepared from commercially available reagents using conventional reactions well known in the art.

### Example 21

### 2-(3,5-dichlorophenyl)-N-phenyl-1H-indole-5-carboximidamide

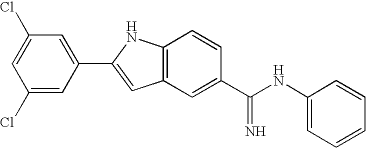

The [2-(3,5-dichlorophenyl)-1H-indol-5-yl](ethoxy)methaniminium chloride (0.076 g, 0.21 mmol) prepared from the second step of example I was taken up in anhydrous THF (2.1 mL), which was then charged with triethylamine (0.057 mL, 0.41 mmol) and aniline (0.023 mL, 0.25 mmol). The reaction mixture was heated at 100° C. for 48 hours, cooled to room temperature, and concentrated. The crude residue was subjected to reverse phase liquid chromatography to provide 2-(3,5-dichlorophenyl)-N-phenyl-1H-indole-5-carboximidamide as a brown solid that gave a mass ion (ES+) of 380.1 for M+H⁺ and proton NMR spectra consistent with theory. ¹H NMR (500 MHz, CD₃OD-d₄) δ 8.24 (s, 1H), 7.85 (s, 1H), 7.85 (s, 1H), 7.66 (m, 2H), 7.62-7.59 (m, 2H), 7.50-7.49 (m, 2H), 7.45 (m, 2H), 7.18 (s, 1H).

TABLE 3
5-Substituted-carboximidamides

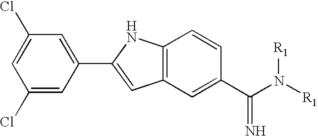

| EXAMPLE | 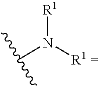 | MASS SPEC (M + H)⁺ |
|---|---|---|
| 22 | NHCH₂PH | 394.1 |
| 23 | NHN-BU | 360.0 |
| 24 | N(CH₃)₂ | 332.1 |
| 25 | 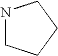 | 358.1 |
| 26 | 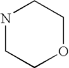 | 318.1 |
| 27 | NHCH₃ | 380.1 |

### Example 28

### (2-Phenyl-1H-indole-3,5-diyl)dimethanamine

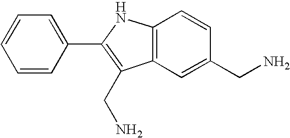

tert-Butyl 5-cyano-2-phenyl-1H-indole-1-carboxylate was prepared according to the Suzuki coupling procedure described in example I, step 1, starting from 1-Boc-5-cyanoindole-2-boronic acid (2.00 g, 6.99 mmol) and iodobenzene (2.85 g, 13.98 mmol). The pure compound gave proton NMR spectra consistent with theory.

To a solution of tert-butyl 5-cyano-2-phenyl-1H-indole-1-carboxylate (450 mg, 1.41 mmol) in DMF (5 mL) was added N-bromo-succinimide (252 mg, 1.41 mmol) at room temperature and the mixture was stirred for 1 hour. The mixture was then partitioned between EtOAc and water. The organic layer was washed with brine, dried over anhydrous magnesium sulfate, filtered and concentrated. The residue was purified by silica gel chromatography with 0 to 30% EtOAc in hexanes to provide tert-butyl 3-bromo-5-cyano-2-phenyl-1H-indole-1-carboxylate that gave a mass ion (ES+) of 399 for M+H⁺ and proton NMR spectra consistent with theory.

To a solution of tert-butyl 3-bromo-5-cyano-2-phenyl-1H-indole-1-carboxylate (100 mg, 0.25 mmol) in anhydrous NMP (1 mL) was added sodium cyanide (25 mg, 0.50 mmol) and nickel bromide (55 mg, 0.25 mmol) and the mixture was heated in a microwave at 200° C. for 30 minutes. It was then partitioned between EtOAc and water. The organic layer was dried over anhydrous MgSO₄, filtered and concentrated. The crude residue was purified by silica gel chromatography to provide tert-butyl 3,5-dicyano-2-phenyl-1H-indole-1-carboxylate that gave a proton NMR spectra consistent with theory.

tert-Butyl 3,5-dicyano-2-phenyl-1H-indole-1-carboxylate (70 mg, 0.20 mmol) was dissolved in a 2M solution of ammonia in methanol (0.5 mL) and diluted with THF (1 mL). The solution was charged with Raney Nickel, flushed with hydrogen three times, and stirred under a hydrogen balloon for 1 hour. The mixture was then filtered through a pad of celite and washed with MeOH. The filtrate was concentrated and the residue was subjected to reverse phase liquid chromatography to provide tert-butyl 3,5-diaminomethyl-2-phenyl-1H-indole-1-carboxylate that gave a mass ion (ES+) of 336 for [M+H]⁺−17 and a proton NMR spectra consistent with theory.

A solution of tert-butyl 3,5-diaminomethyl-2-phenyl-1H-indole-1-carboxylate (25 mg, 0.07 mmol) in EtOAc (50 mL) was saturated with anhydrous HCl gas at room temperature and stirred for 30 minutes. The mixture was then concentrated and the residue was subjected to reverse phase liquid chromatography to provide (2-phenyl-1H-indole-3,5-diyl)dimethanamine that gave a proton NMR spectra consistent with theory. ¹H NMR (400 MHz, CD₃OH) δ 7.68 (m, 2H), 7.61-7.50 (m, 5H), 7.43 (d, J=7.57 Hz, 1H), 4.55 (s, 2H), 4.38 (s, 2H)

TABLE 4
3-Substituted 1H-indole-6-carboximidamides

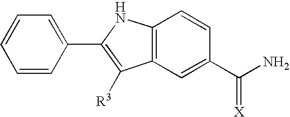

| | | | MASS |
|---|---|---|---|
| | | | SPEC |
| EXAMPLE | R³ = | X = | (M + H)⁺ |
| 29 | CL | NH | 271.2 |
| 30 | CL | H, H | 240.2^{A} |
| 31 | ME | H, H | 220.3^{A} |
| 32 | CH₂NHCH(CH₃)₂ | H, H | 294.3 |
| 33 | CH₂NH(CH₃) | H, H | 249.3^{A} |
| 34 | CH₂NHC-HEXYL | H, H | 334.3 |
| 35 | S(O)PH | H, H | 346.1 |
| 36 | 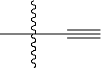 | H, H | 247.2 |
| 37 | SPH | H, H | 314.1^{A} |
| 38 | OME | H, H | 252.2 |
| 39 | ET | H, H | 234.3^{A} |
| 40 | NH₂ | H, H | 220.3^{A} |
| 41 | 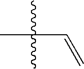 | H, H | 232.3^{A} |
| 42 | C-PR | H, H | 246.2^{A} |
| 43 | OCH(CH₃)₂ | H, H | 262.3^{A} |
| 44 | OH | H, H | 222.1^{A} |
| 45 | CN | H, H | 231.3^{A} |
| 46 | PH | H, H | 282.2^{A} |
| 47 | BR | H, H | 286.1^{A} |
| 48 | F | NH | 254.3 |
| 49 | CO₂H | H, H | 250.3^{A} |

^{a}Indicates (M − 17)⁺ observed.

### Example 50

### 1-[2-(2,6-Dichloropyridin-4-yl)-1H-indol-5-yl]methanamine

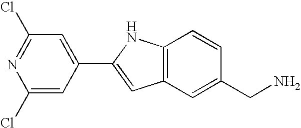

1-(1H-indol-5-yl)methanamine was prepared according to the Raney Nickel hydrogenation procedure described in example 2, step 4, starting from 1H-indole-5-carbonitrile (5.00 g, 35.17 mmol). The reaction was performed overnight. The pure compound gave a mass ion (ES+) of 293 for M+H⁺.

To a solution of 1-(1H-indol-5-yl)methanamine (4.00 g, 27.3 mmol) in acetonitrile (80 mL) at 0° C. was added di-tert-butyl dicarbonate (13.14 g, 60.18 mmol) and DMAP (336 mg, 2.73 mmol) and the reaction mixture was stirred at room temperature overnight. The reaction mixture was concentrated and the crude residue was purified by silica gel chromatography with 0 to 15% EtOAc in hexanes to provide tert-butyl [(1-Boc-1H-indol-5-yl)methyl]carbamate that gave a mass ion (ES+) of 347 for M+H⁺ and proton NMR spectra consistent with theory.

To a solution of tent-butyl [(1-Boc-1H-indol-5-yl)methyl]carbamate (2.5 g, 7.22 mmol) in THF (20 mL) was added triisopropyl borate (252 mg, 1.41 mmol) and the solution was cooled to −40° C. LDA (8.82 mL of 1.8M solution, 15.88 mmol) was then added dropwise over 1 hour. The reaction was quenched with 1 N HCl (40 mL), warmed to 0° C. and stirred for 20 minutes. The pH was adjusted to 7 by adding a aqueous solution of saturated NH₄OH (8 mL) at 0° C. The biphasic solution was then stirred at room temperature for 30 minutes to ensure all the solids are dissolved. The layers were separated and the organic phase was dried over anhydrous MgSO₄, filtrated and concentrated to provide tert-butyl [(1-Boc-1H-indol-5-yl]methyl]carbamate-2-boronic acid with a mass ion (ES+) of 391 for M+H⁺ and proton NMR spectra consistent with theory.

tert-Butyl {[1-Boc-2-2,6-dichloropyridin-4-yl)-1H-indol-5-yl]methyl}carbamate was prepared according the Suzuki coupling procedure described in example 1, step 1, starting from tert-butyl [(1-Boc-1H-indol-5-yl)methyl]carbamate-2-boronic acid (50 mg, 0.13 mmol) and 2,6-dichloro-4-iodopyridine (36 mg, 0.13 mmol).

1-[2-(2,6-dichloropyridin-4-yl)-1H-indol-5-yl]methanamine was prepared according the Boc-deprotecting procedure described in example 2, step 5, starting from tert-butyl {[1-Boc-2-2,6-dichloropyridin-4-yl)-1H-indol-5-yl]methyl}carbamate. It gave a mass ion (ES+) of 276 for [M+H]⁺−17.

TABLE 5
2-Substituted 1-(1H-indole-5-yl)methanamines

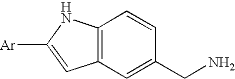

| | | MASS |
|---|---|---|
| | | SPEC (M − |
| EXAMPLE | AR = | 17)⁺ |
| 51 | 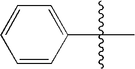 | 206.1 |
| 52 | 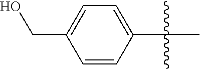 | 236.2 |
| 53 | 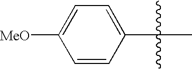 | 236.1 |
| 54 | 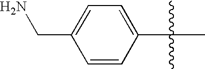 | |
| 55 | 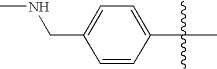 | 249.3 |
| 56 | 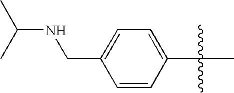 | 277.3 |
| 57 | 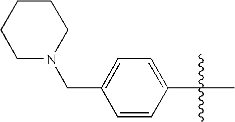 | 303.3 |
| 58 | 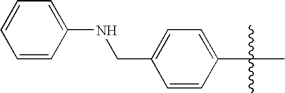 | 311.2 |
| 59 | 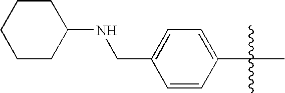 | 334.2 |
| 60 | 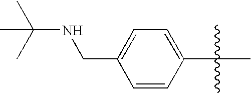 | 308.2 |
| 61 | 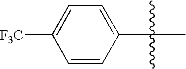 | 274.1 |
| 62 | 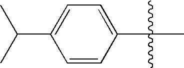 | 248.1 |
| 63 | 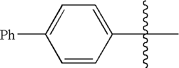 | 282.1 |
| 64 | 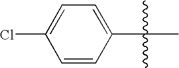 | 240.1 |
| 65 | 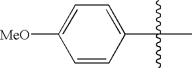 | 236.1 |
| 66 | 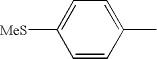 | 252.1 |
| 67 | 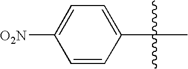 | 251.1 |
| 68 | 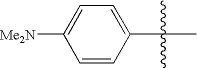 | 249.1 |
| 69 | 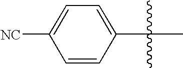 | 231.1 |
| 70 | 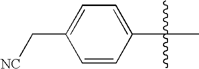 | 245.1 |
| 71 | 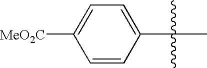 | 264.1 |
| 72 | 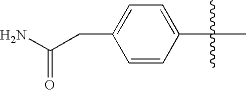 | 245.1 |
| 73 | 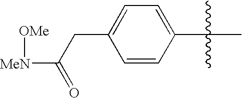 | 307.1 |
| 74 | 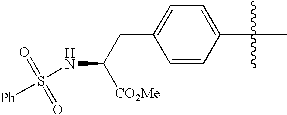 | 448.1 |
| 75 | 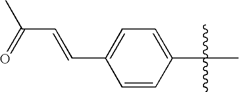 | 290.1 |
| 76 | 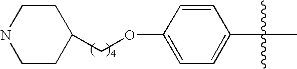 | 361.2 |
| 77 | 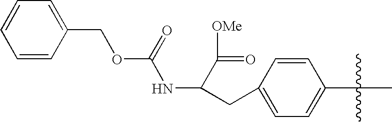 | 441.2 |
| 78 | 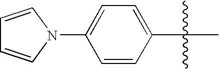 | 271.1 |
| 79 | 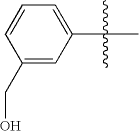 | 236.3 |
| 80 | 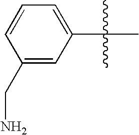 | 235.1 |
| 81 | 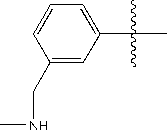 | 249.2 |
| 82 | 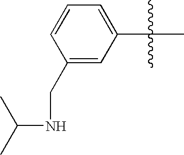 | 294.3 |
| 83 | 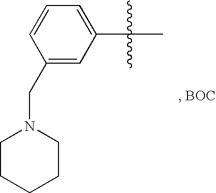 | 420.2 |
| 84 | 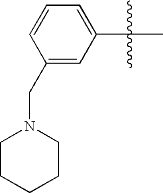 | 320.2 |
| 85 | 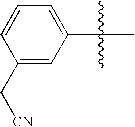 | 245.1 |
| 86 | 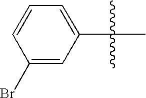 | 284.0 |
| 87 | 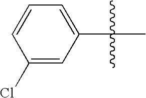 | 240.1 |
| 88 | 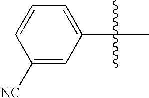 | 231.1 |
| 89 | 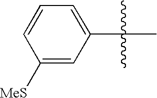 | 252.1 |
| 90 | 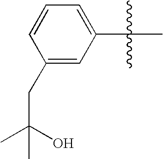 | 278.2 |
| 91 | 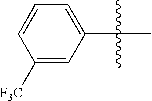 | 274.2 |
| 92 | 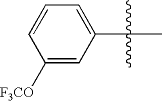 | 290.1 |
| 93 | 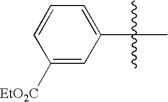 | 278.1 |
| 94 | 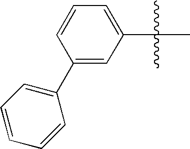 | 282.1 |
| 95 | 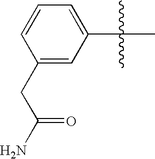 | 245.1 |
| 96 | 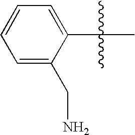 | 235.1 |
| 97 | 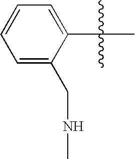 | 249.3 |
| 98 | 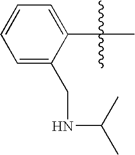 | 294.2 |
| 99 | 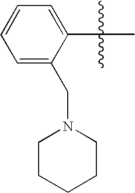 | 320.2 |
| 100 | 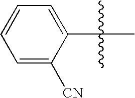 | 231.1 |
| 101 | 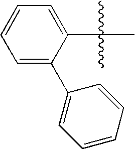 | 282.1 |
| 102 | 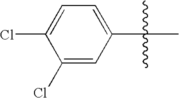 | 274.0 |
| 103 | 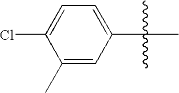 | 254.1 |
| 104 | 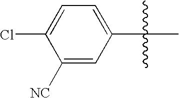 | 265.1 |
| 105 | 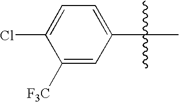 | 308.1 |
| 106 | 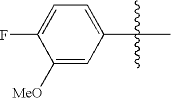 | 254.1 |
| 107 | 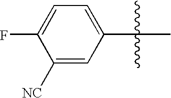 | 249.1 |
| 108 | 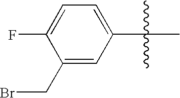 | 316.0 |
| 109 | 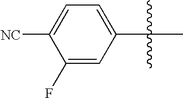 | 249.1 |
| 110 | 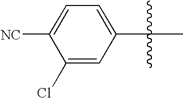 | 265.1 |
| 111 | 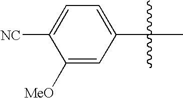 | 261.1 |
| 112 | 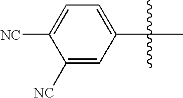 | 256.1 |
| 113 | 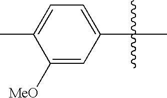 | 250.1 |
| 114 | 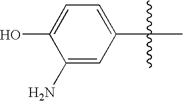 | 237.1 |
| 115 | 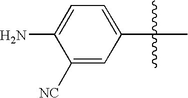 | 246.1 |
| 116 | 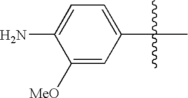 | 251.1 |
| 117 | 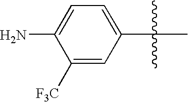 | 289.1 |
| 118 | 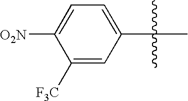 | 319.1 |
| 119 | 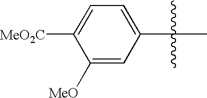 | 294.1 |
| 120 | 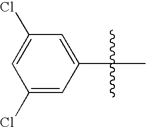 | 274.1 |
| 121 | 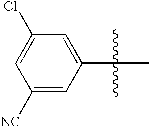 | 267.1 |
| 122 | 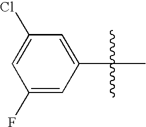 | 258.1 |
| 123 | 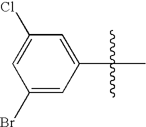 | 317.9 |
| 124 | 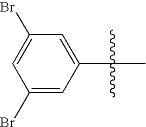 | 361.9 |
| 125 | 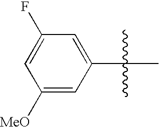 | 254.1 |
| 126 | 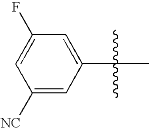 | 249.1 |
| 127 | 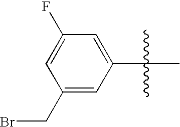 | 316.0 |
| 128 | 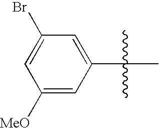 | 314.0 |
| 129 | 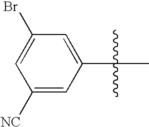 | 311.1 |
| 130 | 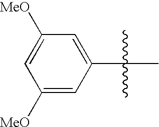 | 266.1 |
| 131 | 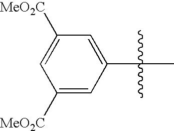 | 308.1 |
| 132 | 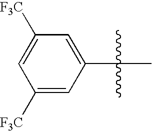 | 342.1 |
| 133 | 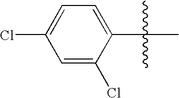 | 274.0 |
| 134 | 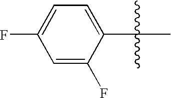 | 242.1 |
| 135 | 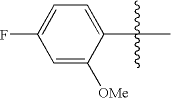 | 254.1 |
| 136 | 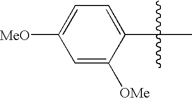 | 266.1 |
| 137 | 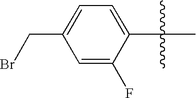 | 316.1 |
| 138 | 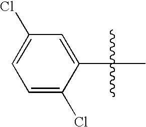 | 274.0 |
| 139 | 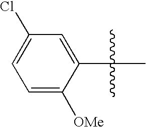 | 270.1 |
| 140 | 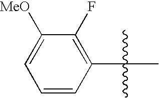 | 254.1 |
| 141 | 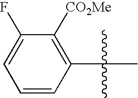 | 282.1 |
| 142 | 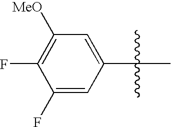 | 272.1 |
| 143 | 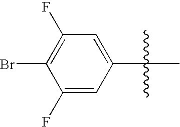 | 320.0 |
| 144 | 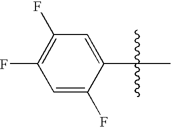 | 260.1 |
| 145 | 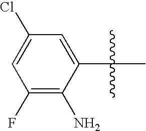 | 273.1 |
| 146 | 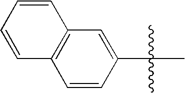 | 256.1 |
| 147 | 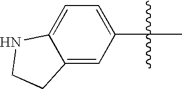 | 247.1 |
| 148 | 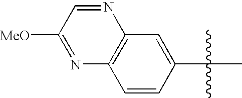 | 288.1 |
| 149 | 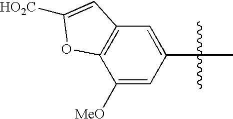 | 320.1 |
| 150 | 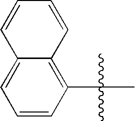 | 256.1 |
| 151 | 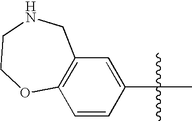 | 277.1 |
| 152 | 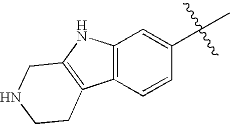 | 300.1 |
| 153 | 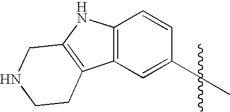 | 300.1 |
| 154 | 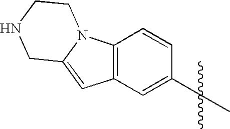 | 300.1 |
| 155 | 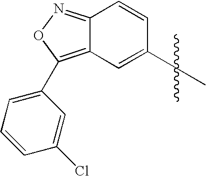 | 359.1 |
| 156 | 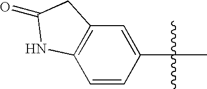 | 261.1 |
| 157 | 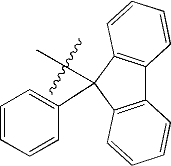 | 370.1 |
| 158 | 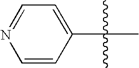 | 224.1 |
| 159 | 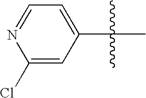 | 241.1 |
| 160 | 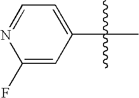 | 225.1 |
| 161 | 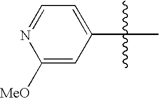 | 254.1H |
| 162 | 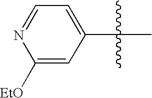 | 251.1 |
| 163 | 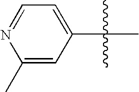 | 221.1 |
| 164 | 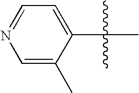 | 221.1 |
| 165 | 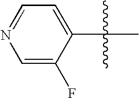 | 225.1 |
| 166 | 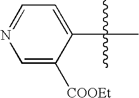 | 225.1 |
| 167 | 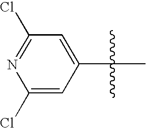 | 275.0 |
| 168 | 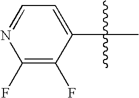 | 243.1 |
| 169 | 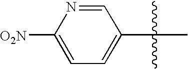 | 252.1 |
| 170 | 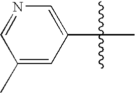 | 221.1 |
| 171 | 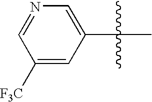 | 275.2 |
| 172 | 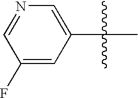 | 242.2H |
| 173 | 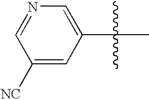 | 232.1 |
| 174 | 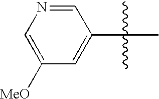 | 254.1L |
| 175 | 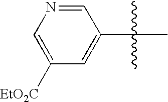 | 279.1 |
| 176 | 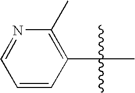 | 221.1 |
| 177 | 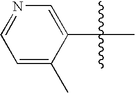 | 221.1 |
| 178 | 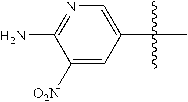 | 267.1 |
| 179 | 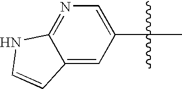 | 246.1 |
| 180 | 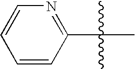 | 224.1L |
| 181 | 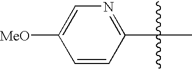 | 254.1L |
| 182 | 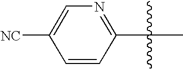 | 232.1 |
| 183 | 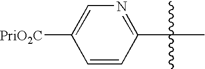 | 293.1 |
| 184 | 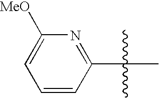 | 237.1 |
| 185 | 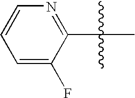 | 225.1 |
| 186 | 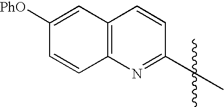 | 349.1 |
| 187 | 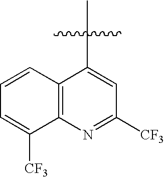 | 393.1 |
| 188 | 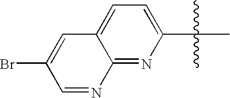 | 336.0 |
| 189 | 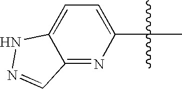 | 247.1 |
| 190 | 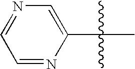 | 208.1 |
| 191 | 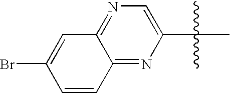 | 336.0 |
| 192 | 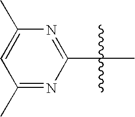 | 236.1 |
| 193 | 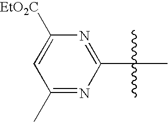 | 294.1 |
| 194 | 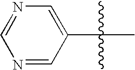 | 208.1 |
| 195 | 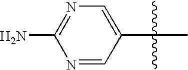 | 223.2 |
| 196 | 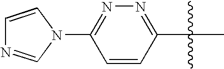 | 274.1 |
| 197 | 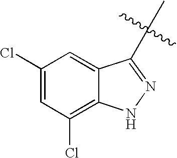 | 315.0 |
| 198 | 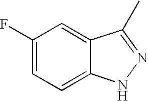 | 264.1 |
| 199 | 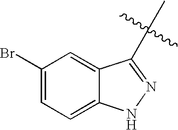 | 341.0 |
| 200 | 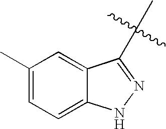 | 277.1 |
| 201 | 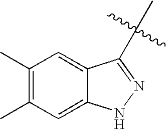 | 291.1 |
| 202 | 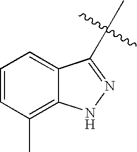 | 277.1 |
| 203 | 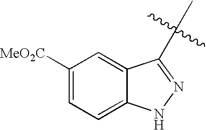 | 304.1 |
| 204 | 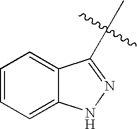 | 263.1 |
| 205 | 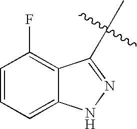 | 281.1 |
| 206 | 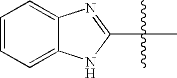 | 263.1 |
| 207 | 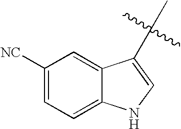 | 271.0 |
| 208 | 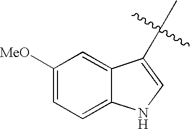 | 292.2 |
| 209 | 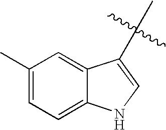 | 276.3 |
| 210 | 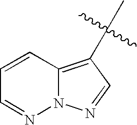 | 264.1 |
| 211 | 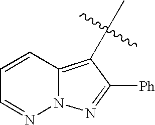 | 341.1 |
| 212 | 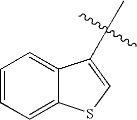 | 279.0 |
| 213 | 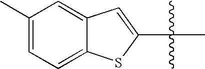 | 293.1 |
| 214 | 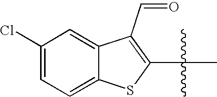 | 324.0 |
| 215 | 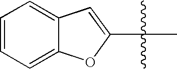 | 263.1 |
| 216 | 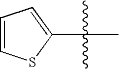 | 229.1 |
| 217 | 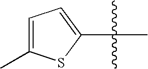 | 243.1 |
| 218 | 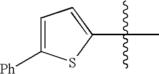 | 305.1 |
| 219 | 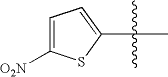 | 274.1 |
| 220 | 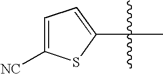 | 254.1 |
| 221 | 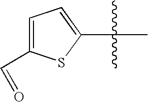 | 257.1 |
| 222 | 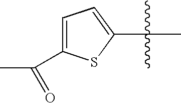 | 271.1 |
| 223 | 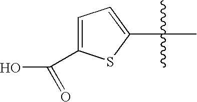 | 273.2 |
| 224 | 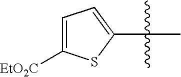 | 301.1 |
| 225 | 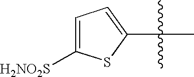 | 308.1 |
| 226 | 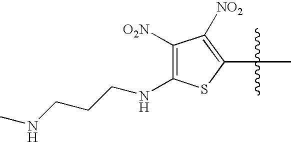 | 419.1 |
| 227 | 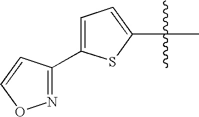 | 296.1 |
| 228 | 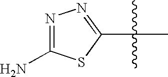 | 246.1 |
| 229 | 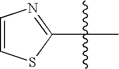 | 230.1 |
| 230 | 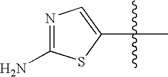 | 245.1 |
| 231 | 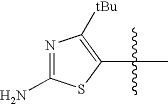 | 301.1 |
| 232 | 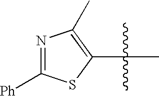 | 320.1 |
| 233 | 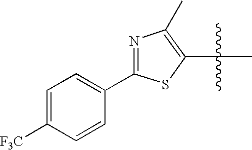 | 388.1 |
| 234 | 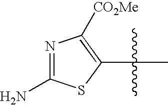 | 303.1 |
| 235 | 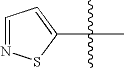 | 230.1 |
| 236 | 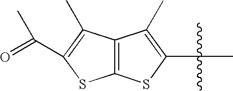 | 355.1 |
| 237 | 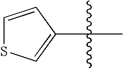 | 229.1 |
| 238 | 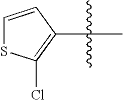 | 263.1 |
| 239 | 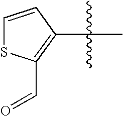 | 257.1 |
| 240 | 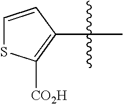 | 273.1 |
| 241 | 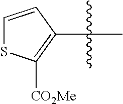 | 287.1 |
| 242 | 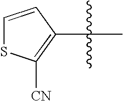 | 254.1 |
| 243 | 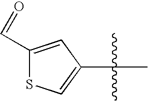 | 257.1 |
| 244 | 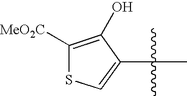 | 303.1 |
| 245 | 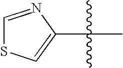 | 213.1 |
| 246 | 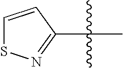 | 230.1 |
| 247 | 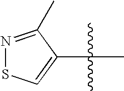 | 244.1 |
| 248 | 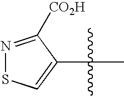 | 273.1 |
| 249 | 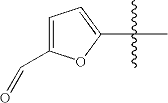 | 241.1 |
| 250 | 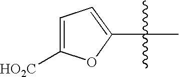 | 257.1 |
| 251 | 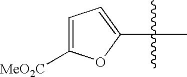 | 270.1 |
| 252 | 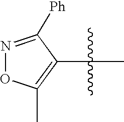 | 304.1 |
| 253 | 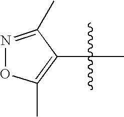 | 242.1 |
| 254 | 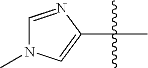 | 227.1 |
| 255 | 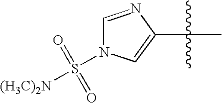 | 320.1 |
| 256 | 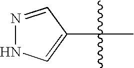 | 213.1 |
| 257 | 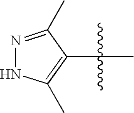 | 241.1 |
| 258 | 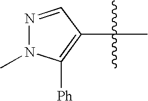 | 303.1 |
| 259 | 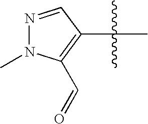 | 255.1 |
| 260 | 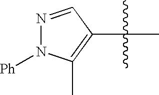 | 303.1 |
| 261 | 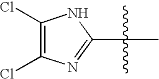 | 282.1 |

What is claimed is:

1. A compound according to Formula I or Formula II 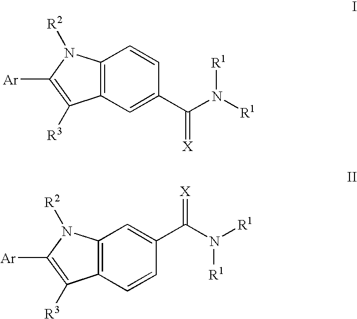
or a pharmaceutically acceptable salt of a compound of Formula I or Formula II, wherein:
X=NH or H,H
each R¹ is independently selected from the group consisting of: H, —OR², —CO₂R², —CON(R²)₂, C₁₋₄alkyl, C₁₋₄alkyloxy, phenyl, benzyl, —CN and —COR²; or the two R¹ groups can be joined together with the nitrogen atom to which they are attached to form a 5- or 6-membered monocyclic ring, optionally containing a heteroatom selected from O, S or N;
each R² is independently selected from the group consisting of: H, C₁₋₈ alkyl, C₃₋₈ cycloalkyl and phenyl;
R³ is selected from the group consisting of: H, C₁₋₈alkyl, C₂₋₈alkenyl, C₂₋₈alkynyl, C₃₋₈cycloalkyl, halogen, C₁₋₄alkyloxy, —CN, —NO₂, —N(R⁵)₂, —NR⁵COR⁴, —CO₂R⁴, —COR⁴, —OH, —SR⁴, —SOR⁴, —SO₂R⁴, —SO₂NHR⁵, —NONHR⁵ and phenyl, wherein said C₁₋₈alkyl is optionally substituted with 1 to 3 substituents independently selected from the group consisting of: halogen, OR⁴, nitro, cyano, C₁₋₄alkyloxy, COR⁴, SO₂R⁴ and CO₂R⁴; and
Ar is aryl or heteroaryl, each optionally substituted with 1 to 5 R^{a} groups, wherein each R^{a} is independently selected from the group consisting of: halogen, —OR⁴, —S—R⁴, —COR⁴, —NO₂, —N(R⁵)₂, C₁₋₄alkyl, C₁₋₄haloalkyl, C₁₋₄hydroxyalkyl, aryl, heteroaryl, N(R⁵)₂—CH₂—, N(R⁵)₂—SO₂—, —CN, CN—CH₂—, —CF₃, N(R⁵)₂—C(O)—CH₂—, R⁴—SO₂—N(H)—CH(R³)—CH₂—, CH₃—C(O)—CH═CH—, heterocyclyl-C₁₋₄-alkylenyl-O—, R⁴—O—C(O)—NH—CH(R³)—CH₂—, R⁵—N(H)—(CH₂)₃—N(H)— and phenyl substituted with 1 to 5 substituents independently selected from the group consisting of: halo, C₁₋₄alkyl and C₁₋₄haloalkyl;
each R⁴ is independently selected from the group consisting of: H, C₁₋₈ alkyl, C₁₋₄haloalkyl, C₃₋₈ cycloalkyl, phenyl and benzyl; and
each R⁵ is independently selected from the group consisting of: H, C₁₋₈ alkyl, C₃₋₈ cycloalkyl, phenyl, benzyl, OR² or two R⁵ groups can be joined together with the nitrogen atom to which thy are attached to form a six-membered monocyclic ring.

2. The compound according to claim 1 of Formula I.

3. The compound according to claim 2 wherein R² is H.

4. The compound according to claim 2 wherein X is NH.

5. The compound according to claim 2 wherein X is H,H.

6. The compound according to claim 2 wherein Ar is phenyl, optionally substituted with 1 to 5 R^{a} groups.

7. The compound according to claim 2 wherein Ar is pyridyl, optionally substituted with 1 to 5 R^{a} groups.

8. The compound according to claim 2 of Formula Ia 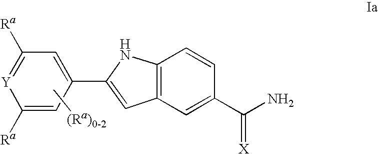
or a pharmaceutically acceptable salt thereof, wherein
X=NH or H,H;
Y is CH or N;
each R^{a} is independently selected from the group consisting of: halogen, —OR⁴, —S—R⁴, —CO₂R⁴, —COR⁴, —NO₂, —N(R⁵)₂, C₁₋₄alkyl, C₁₋₄haloalkyl, C₁₋₄hydroxyalkyl, aryl, heteroaryl, N(R⁵)₂—CH₂—, N(R⁵)₂—SO₂—, —CN, CN—CH₂—, —CF₃, N(R⁵)₂—C(O)—CH₂—, R⁴—SO₂—N(H)—CH(R³)—CH₂—, CH₃—C(O)—CH═CH—, heterocyclyl-C₁₋₄-alkylenyl-O—, R⁴—O—C(O)—NH—CH(R³)—CH₂—, R⁵—N(H)—(CH₂)₃—N(H)— and phenyl substituted with 1 to 5 substituents independently selected from the group consisting of: halo, C₁₋₄alkyl and C₁₋₄haloalkyl;
each R⁴ is independently selected from the group consisting of: H, C₁₋₈ alkyl, C₁₋₄haloalkyl, C₃₋₈ cycloalkyl, phenyl and benzyl; and
each R⁵ is independently selected from the group consisting of: H, C₁₋₈ alkyl, C₃₋₈ cycloalkyl, phenyl, benzyl, OR² or two R⁵ groups can be joined together with the nitrogen atom to which thy are attached to form a six-membered monocyclic ring.

9. The compound according to claim 8 wherein each R^{a} is independently selected from the group consisting of: HO—CH₂—, F, Cl, Br, NH₂, CH₃—O—, —CN, —CH₃, phenyl, phenylamino, benzylamino, formyl and 1-piperidinylmethyl.

10. The compound according to claim 9 wherein X is NH.

11. The compound according to claim 9 wherein X is H,H.

12. A compound according to claim 1 selected from the following: 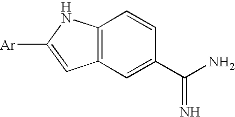
AR = 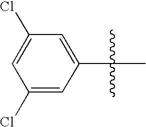 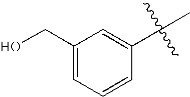 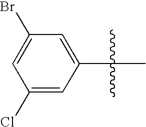 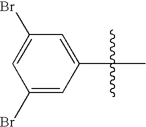 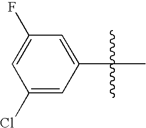 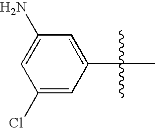 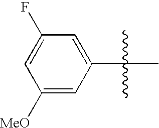 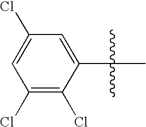 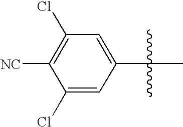 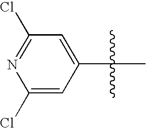 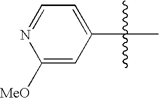 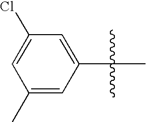 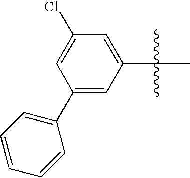 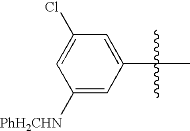 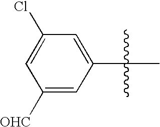 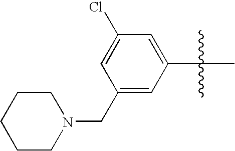 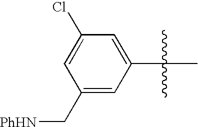
or a pharmaceutically acceptable salt of any of the foregoing compounds.

13. A compound according to claim 1 selected from the following: 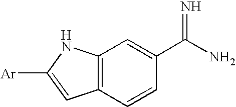
AR =

| | | |
|---|---|---|
| 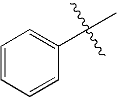 | 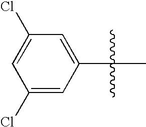 | 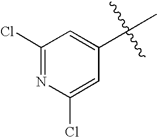 |

or a pharmaceutically acceptable salt of any of the foregoing compounds.

14. A compound according to claim 1 selected from the following:

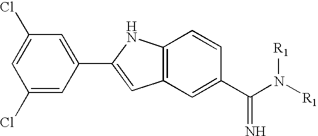

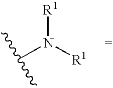

| | | | |
|---|---|---|---|
| NHPH | NHCH₂PH | NHN-BU | N(CH₃)₂ |
| | | |
|---|---|---|
| 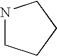 | 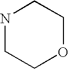 | NHCH₃ |

or a pharmaceutically acceptable salt of any of the foregoing compounds.

15. A compound according to claim 1 selected from the following:

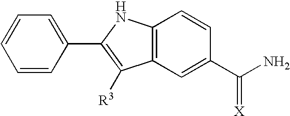

| R³ = | X = |
|---|---|
| CH₂NH₂ | H, H |
| CL | NH |
| CL | H, H |
| ME | H, H |
| S(O)PH | H, H |
| 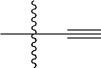 | H, H |
| SPH | H, H |
| OME | H, H |
| ET | H, H |
| NH₂ | H, H |
| 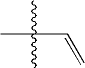 | H, H |
| C-PR | H, H |
| OH | H, H |
| CN | H, H |
| PH | H, H |
| BR | H, H |
| F | NH |
| CO₂H | H, H |

or a pharmaceutically acceptable salt of any of the foregoing compounds.

16. A compound according to claim 1 selected from the following: 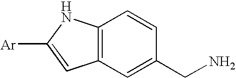
AR =

| | |
|---|---|
| 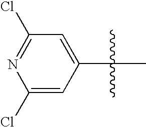 | 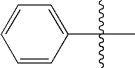 |
| 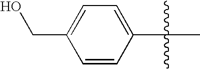 | 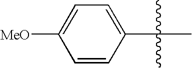 |
| | |
|---|---|
| 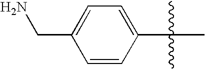 | 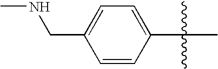 |

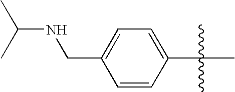

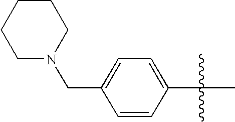

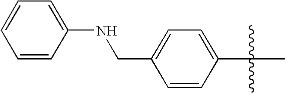

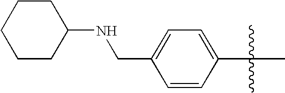
| | |
|---|---|
| 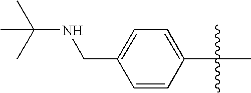 | 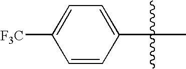 |
| | |
|---|---|
| 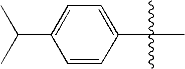 | 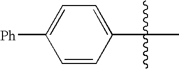 |
| 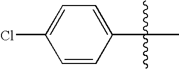 | 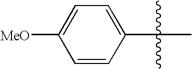 |
| 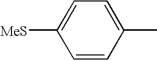 | 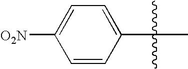 |
| | |
|---|---|
| 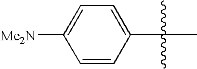 | 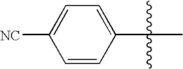 |
| | |
|---|---|
| 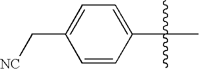 | 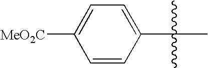 |

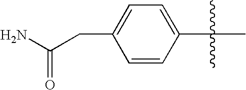

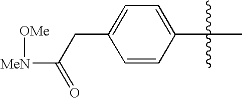

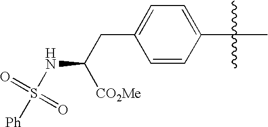

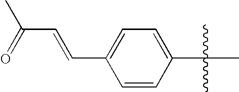

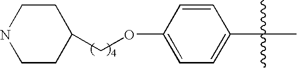

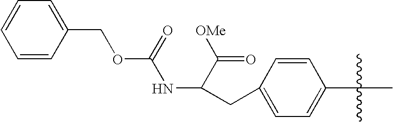
| | |
|---|---|
| 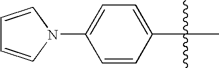 | 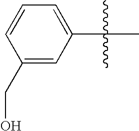 |
| | |
|---|---|
| 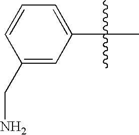 | 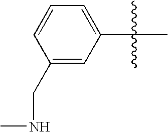 |
| 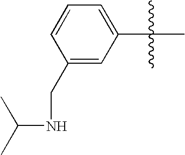 | 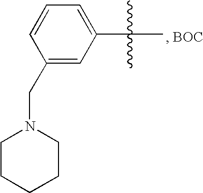 |
| | |
|---|---|
| 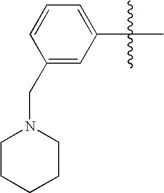 | 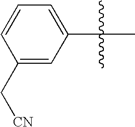 |
| | |
|---|---|
| 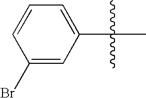 | 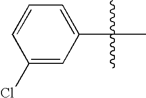 |
| 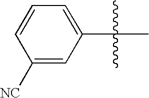 | 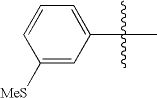 |
| 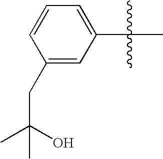 | 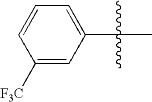 |
| 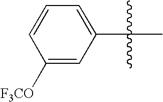 | 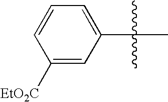 |
| | |
|---|---|
| 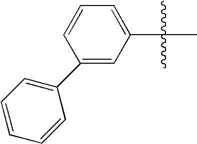 | 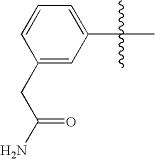 |
| | | |
|---|---|---|
| 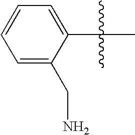 | 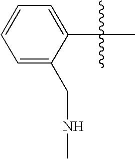 | 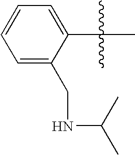 |
| 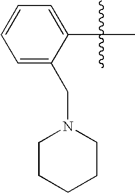 | 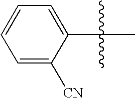 | 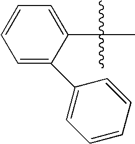 |
| | |
|---|---|
| 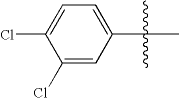 | 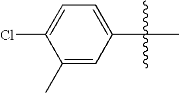 |
| 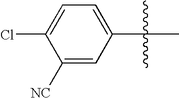 | 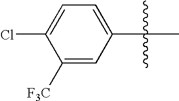 |
| 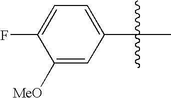 | 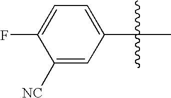 |
| 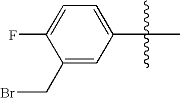 | 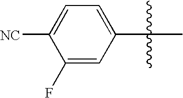 |
| 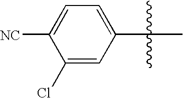 | 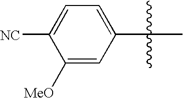 |
| 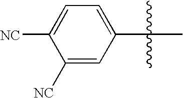 | 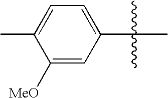 |
| | |
|---|---|
| 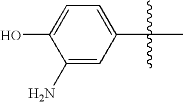 | 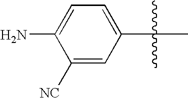 |
| | |
|---|---|
| 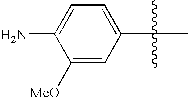 | 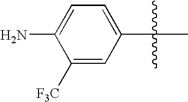 |
| | |
|---|---|
| 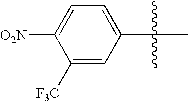 | 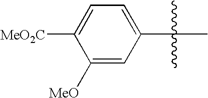 |
| | |
|---|---|
| 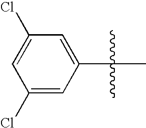 | 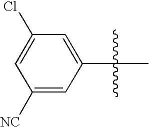 |
| 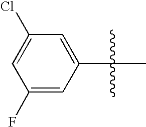 | 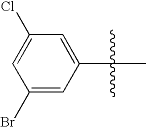 |
| 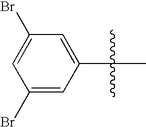 | 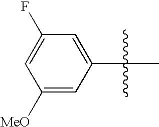 |
| | |
|---|---|
| 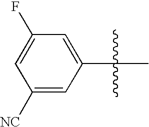 | 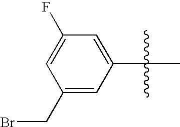 |
| | |
|---|---|
| 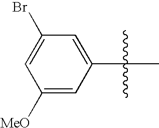 | 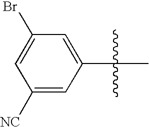 |
| | |
|---|---|
| 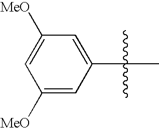 | 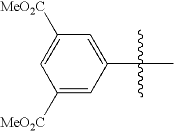 |
| 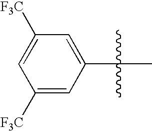 | 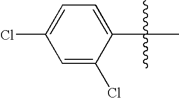 |
| | |
|---|---|
| 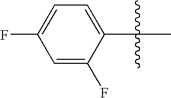 | 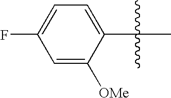 |
| 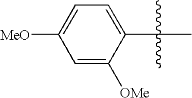 | 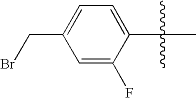 |
| 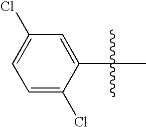 | 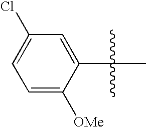 |
| 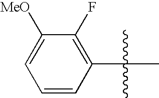 | 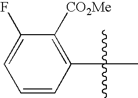 |
| | |
|---|---|
| 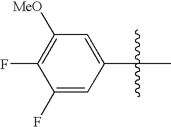 | 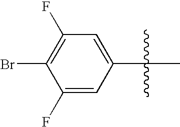 |
| | |
|---|---|
| 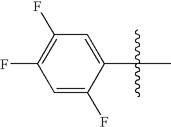 | 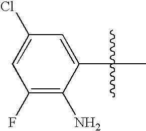 |
| 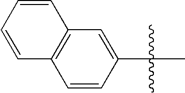 | 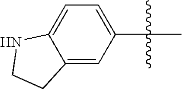 |

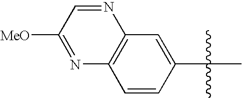
| | |
|---|---|
| 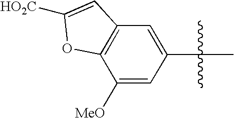 | 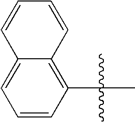 |
| | |
|---|---|
| 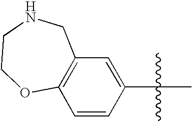 | 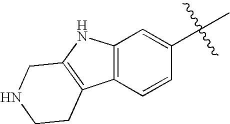 |

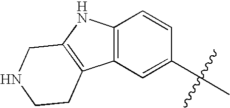
| | |
|---|---|
| 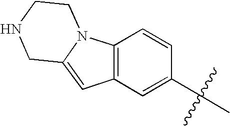 | 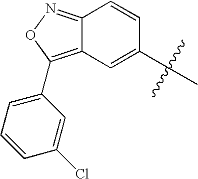 |
| | |
|---|---|
| 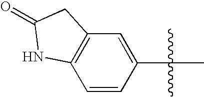 | 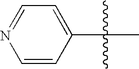 |
| | |
|---|---|
| 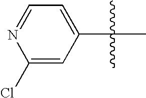 | 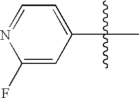 |
| | |
|---|---|
| 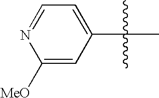 | 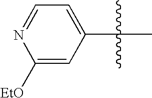 |
| | | |
|---|---|---|
| 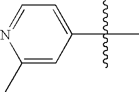 | 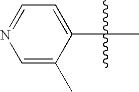 | 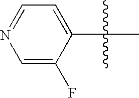 |
| | | |
|---|---|---|
| 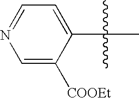 | 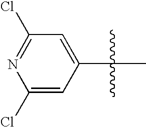 | 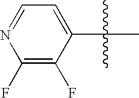 |
| | |
|---|---|
| 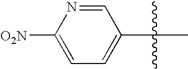 | 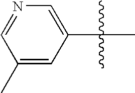 |
| | |
|---|---|
| 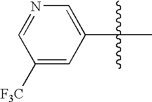 | 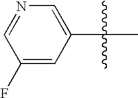 |
| | |
|---|---|
| 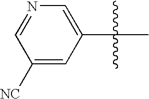 | 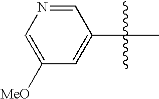 |
| | |
|---|---|
| 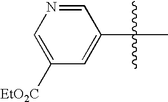 | 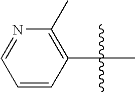 |
| | |
|---|---|
| 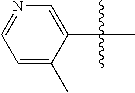 | 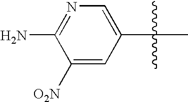 |
| | |
|---|---|
| 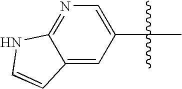 | 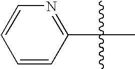 |
| | |
|---|---|
| 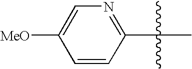 | 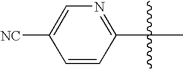 |
| | |
|---|---|
| 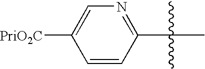 | 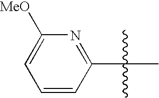 |
| | |
|---|---|
| 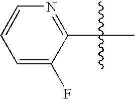 | 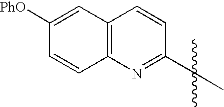 |
| 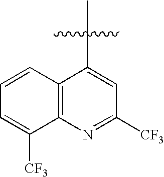 | 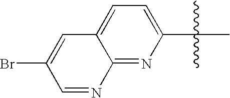 |
| | |
|---|---|
| 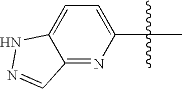 | 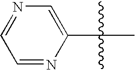 |
| | |
|---|---|
| 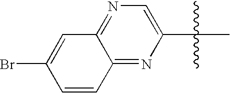 | 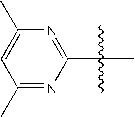 |
| | |
|---|---|
| 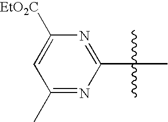 | 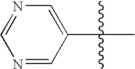 |
| | |
|---|---|
| 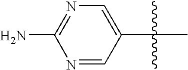 | 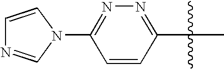 |
| | |
|---|---|
| 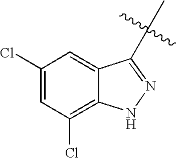 | 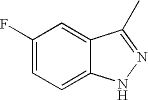 |
| | |
|---|---|
| 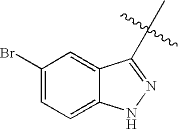 | 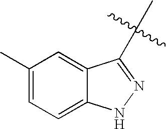 |
| | |
|---|---|
| 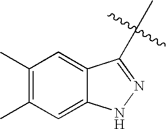 | 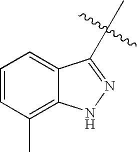 |
| | |
|---|---|
| 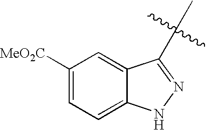 | 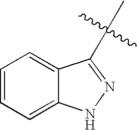 |
| | |
|---|---|
| 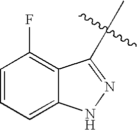 | 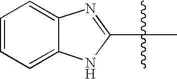 |
| | |
|---|---|
| 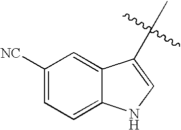 | 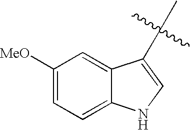 |
| | |
|---|---|
| 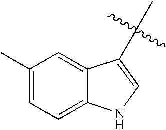 | 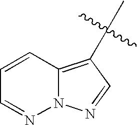 |
| | |
|---|---|
| 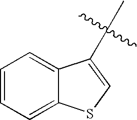 | 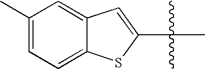 |
| | |
|---|---|
| 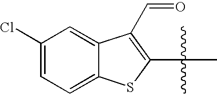 | 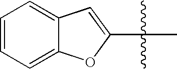 |
| | |
|---|---|
| 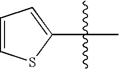 | 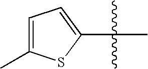 |
| | |
|---|---|
| 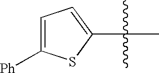 | 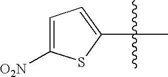 |
| 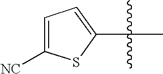 | 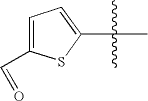 |
| 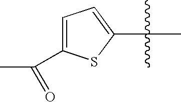 | 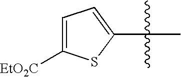 |

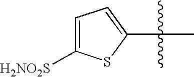

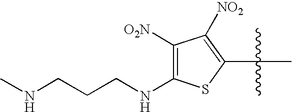
| | |
|---|---|
| 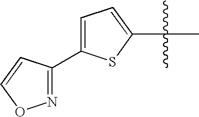 | 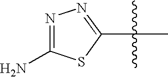 |
| | |
|---|---|
| 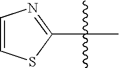 | 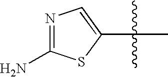 |
| | |
|---|---|
| 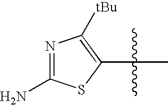 | 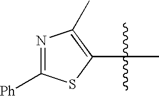 |
| | |
|---|---|
| 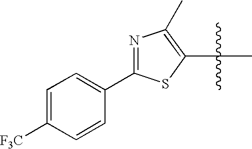 | 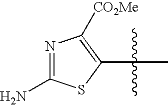 |
| | |
|---|---|
| 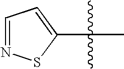 | 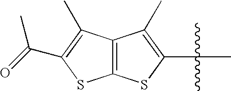 |
| | | |
|---|---|---|
| 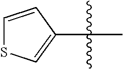 | 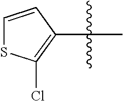 | 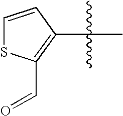 |
| | | |
|---|---|---|
| 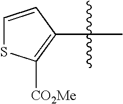 | 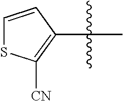 | 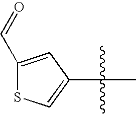 |
| | | |
|---|---|---|
| 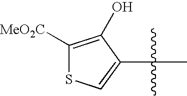 | 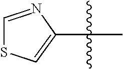 | 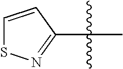 |
| | | |
|---|---|---|
| 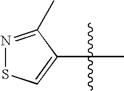 | 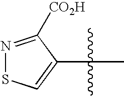 | 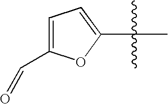 |
| | |
|---|---|
| 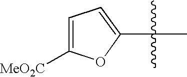 | 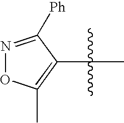 |
| | |
|---|---|
| 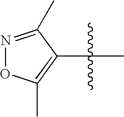 | 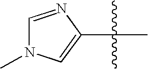 |
| | |
|---|---|
| 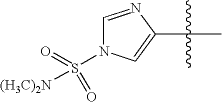 | 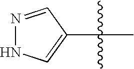 |
| | |
|---|---|
| 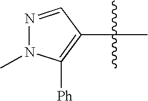 | 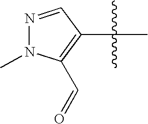 |
| 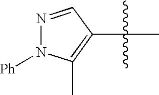 | 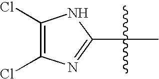 |

or a pharmaceutically acceptable salt of any of the foregoing compounds.

17. A pharmaceutical composition comprising a compound according to claim 1 in combination with a pharmaceutically acceptable carrier.

18. A method for treating pain in a mammalian patient in need thereof comprising administering to the patient a therapeutically effective amount of a compound according to claim 1.

19. A method for treating a disease or condition selected from the group consisting of: (1) chronic, visceral, inflammatory and neuropathic pain syndromes; (2) pain resulting from traumatic nerve injury, nerve compression or entrapment, postherpetic neuralgia, trigeminal neuralgia, and diabetic neuropathy; (3) chronic lower back pain, phantom limb pain, chronic pelvic pain, neuroma pain, complex regional pain syndrome, chronic arthritic pain and related neuralgias, and (4) pain associated with cancer, chemotherapy, HIV and HIV treatment-induced neuropathy, in a mammalian patient in need thereof comprising administering to the patient a therapeutically effective amount of a compound according to claim 1.

20. A method for treating a disease or condition selected from the group consisting of: stroke, anxiety, bone cancer pain, bone disorders with increased osteoclastic bone resorption, ischemic pain, sickle cell anemia, anemia pain, intermittent claudication, gastric mobility disorders, irritable bowel syndrome or disease, inflammatory bowel syndrome or disease and satiety-obesity, in a mammalian patient in need thereof comprising administering to the patient a therapeutically effective amount of a compound according to claim 1.

21. The method according to claim 19 wherein the disease or condition is bone disorders with increased osteoclastic bone resorption selected from the group consisting of: metastatic bone diseases, Paget's disease of bone, osteoporosis, fibrous dysplasia and osteogenesis imperfecta.
